(19) United States
(12) Patent Application Publication
(10) Pub. No.: US 2024/0175041 A1
(43) Pub. Date: May 30, 2024

(54) INFECTIOUS CLONE CAPABLE OF INOCULATING NANOVIRUS DNA-C, DNA-M, DNA-N AND DNA-U1 AND USE THEREOF

(71) Applicant: RESEARCH & BUSINESS FOUNDATION SUNGKYUNKWAN UNIVERSITY, Suwon-si (KR)

(72) Inventors: Suk Chan LEE, Suwon-si (KR); Aamir LAL, Suwon-si (KR)

(73) Assignee: RESEARCH & BUSINESS FOUNDATION SUNGKYUNKWAN UNIVERSITY, Suwon-si (KR)

(21) Appl. No.: 18/523,237

(22) Filed: Nov. 29, 2023

(30) Foreign Application Priority Data
Nov. 29, 2022 (KR) 10-2022-0162595
Nov. 29, 2022 (KR) 10-2022-0162596
May 15, 2023 (KR) 10-2023-0062725
May 15, 2023 (KR) 10-2023-0062726

(51) Int. Cl. C12N 15/82; C12N 7/00; C12Q 1/6806; C12Q 1/686; C12Q 1/70

(52) U.S. Cl. CPC C12N 15/8203; C12N 7/00; C12Q 1/6806; C12Q 1/686; C12Q 1/701; C12N 2750/00043

(57) ABSTRACT

Disclosed herein are an infectious clone capable of inoculating Nanovirus DNA-C, DNA-M, DNA-N, and DNA-U1, and uses thereof. A recombinant vector comprising the nucleotide sequences of Nanovirus DNA-C, DNA-M, DNA-N, and DNA-U1 represented by SEQ ID NOS: 1 to 8, and E. coli and Agrobacterium tumefaciens, each transformed therewith, can effectively induce the infection of Nanovirus into crops even without any insect vectors. Therefore, the disclosure can be applied to various studies, such as investigating the host range and the correlation with hosts of Nanovirus DNA-C, DNA-M, DNA-N, and DNA-U1. This can help in preemptively preventing economic losses due to virus infections, making it beneficially applicable in the related fields.

### CROSS REFERENCE TO RELATED APPLICATIONS

This application claims priority and the benefit of Korean Patent Application No. 10-2022-0162595 filed in the Korean Intellectual Property Office on Nov. 29, 2022, Korean Patent Application No. 10-2022-0162596 filed in the Korean Intellectual Property Office on Nov. 29, 2022, Korean Patent Application No. 10-2023-0062725 filed in the Korean Intellectual Property Office on May 15, 2023, and Korean Patent Application No. 10-2023-0062726 filed in the Korean Intellectual Property Office on May 15, 2023, the entire disclosures of which are incorporated herein by reference for all purposes.

### INCORPORATION-BY-REFERENCE OF MATERIAL SUBMITTED BY U.S.P.T.O. EFS-WEB

The instant application contains a Sequence Listing which is submitted in computer readable form via the United States Patent and Trademark Office eFS-WEB system and which is hereby incorporated by reference in its entirety for all purposes. The XML file submitted herewith is named NewApp_0421930008_20231129LFCE2_PU230039US_Seq.xml. The XML file has a date of creation of Nov. 29, 2023, and the size of the XML file is 145,747 bytes.

### BACKGROUND OF THE INVENTION

### 1. Field of the Invention

The present disclosure relates to an infectious clone capable of inoculating Nanovirus DNA-C, DNA-M, DNA-N, and DNA-U1, and uses thereof.

### 2. Description of the Prior Art

Nanovirus is a plant virus with a single-strand circular DNA as its genome and is known to cause damage to major economic crops of legumes worldwide. Due to the nature of the infected hosts, it was mainly reported in tropical and subtropical regions, and in Korea, the first case of Nanovirus infection was reported in 2017.

With the agreement of international free trade and increased agricultural trade in the 2000s, various pathogens and pests, including those carrying Nanovirus, have been introduced. This situation is exacerbated by global warming, which creates favorable conditions for the spread of these viruses. Therefore, it is necessary to establish a system to prevent introduction and outbreak of viruses by conducting research on plant viruses in advance that could cause significant damage to the country, especially Nanovirus, whose damage has recently been increasing globally.

Nanovirus is known to be transmitted only through specific insect vectors and infected seeds, not mechanically. In Korea, the aphids Acyrthosiphon pisum and Aphis craccivora are known vectors of Nanovirus. The aphids themselves pose a threat to agricultural crops, creating research challenges due to their diminutive size and the complexities involved in controlling and using them for inoculation experiments. Currently, a known method to overcome these challenges and inoculate Nanovirus more easily involves directly inserting infectious clones into plant cells using Agrobacterium tumefaciens.

Nanovirus is currently being reported in various regions. Among them, the milk vetch dwarf virus (MDV) is a type of Nanovirus that has been reported in Japan and China. It infects leguminous plants, causing necrosis and chlorosis, which inhibits plant growth and reduces yield. Similarly, Faba bean necrotic yellows virus (FBNYV), first reported in Middle Eastern countries like Syria, Turkey, Jordan, and Lebanon, is well-known for infecting legumes, leading to plant necrosis, yellowing, stunting. and chlorosis, thereby limiting growth and reducing harvesting.

DNA-C, DNA-M, DNA-N, and DNA-U1 components of Nanovirus (MDV and FBNYV), are known as the primary infectious agents. Specifically, MDV was first detected in 2017 in papayas being cultivated in open fields in Yesan, South Chungcheong Province, and later infections were confirmed in papaya field showing symptoms of MDV in Taiwan and Vietnam. FBNYV is currently spreading to North African countries like Egypt, Morocco, Algeria, and Mediterranean countries like Spain, infecting leguminous crops such as faba beans and lentils, causing significant economic loss due to reduced yields.

Therefore, there is a critical need for research on these viruses, including the construction of infectious clones and the development of an artificial infection system for research purpose.

### RELATED ART DOCUMENT

### Non-Patent Literature

(Non-patent literature 01) T. Timchenko et al., Journal of General Virology. Vol. 87, No. 6, pp. 1735-1743 (2006.)

### SUMMARY OF THE INVENTION

Leading to the present disclosure, intensive and thorough research, conducted by the present inventors to develop an artificial infection system for Nanovirus. This involved constructing infectious clones of Nanovirus DNA-C, DNA-M, DNA-N, and DNA-U1, enabling artificial infection in plants through Agro-inoculation facilitated by a binary expression vector. Therefore, an aspect of the present disclosure is to provide a recombinant vector comprising the Nanovirus nucleotide sequence represented by at least one of SEQ ID NOS: 1 to 8.

In detail, the present disclosure aims to provide a recombinant vector comprising at least one selected from the group consisting of: the nucleotide sequence of Nanovirus DNA-C represented by SEQ ID NO: 1; the nucleotide sequence of Nanovirus DNA-M represented by SEQ ID NO: 2; the nucleotide sequence of Nanovirus DNA-N represented by SEQ ID NO: 3; the nucleotide sequence of Nanovirus DNA-U1 represented by SEQ ID NO: 4; the nucleotide sequence of Nanovirus DNA-C represented by SEQ ID NO: 5; the nucleotide sequence of Nanovirus DNA-M represented by SEQ ID NO: 6; the nucleotide sequence of Nanovirus DNA-N represented by SEQ ID NO: 7; and the nucleotide sequence of Nanovirus DNA-U1 represented by SEQ ID NO: 8; or a recombinant vector comprising a combination of all.

Another aspect of the present disclosure is to provide Agrobacterium tumefaciens comprising the recombinant vector.

A further aspect of the present disclosure is to provide a Nanovirus -infected plant comprising the recombinant vector.

Another aspect of the present disclosure is to provide a composition for diagnosis of Nanovirus infection in plants to detect and identify each component of nanoviruses, wherein the each component of nanoviruses is DNA-C, DNA-M, DNA-N, DNA-U1, or fragments thereof, which comprises the polynucleotide selected from SEQ ID NOS: 1 to 8.

Another aspect of the present disclosure is to provide a method for diagnosis of Nanovirus infection in a plant.

A still further aspect of the present disclosure is to provide a method for inducing a Nanovirus disease in a plant, which includes a step of transfecting the recombinant vector into the plant.

The present inventors have made efforts in research to develop an artificial infection system for studying Nanovirus. As a result, an expression vector was constructed to carry the infectious clones of Nanovirus DNA-C, DNA-M, DNA-N, and DNA-U1 , and then the vector was introduced into plants through Agro-inoculation, to caused successful infection of Nanovirus in plants for evaluating its effects.

In accordance with an aspect thereof, the present disclosure provides a recombinant vector comprising at least one selected from the group consisting of: the nucleotide sequence of Nanovirus DNA-C represented by SEQ ID NO: 1; the nucleotide sequence of Nanovirus DNA-M represented by SEQ ID NO: 2; the nucleotide sequence of Nanovirus DNA-N represented by SEQ ID NO: 3; and the nucleotide sequence of Nanovirus DNA-U1 represented by SEQ ID NO: 4.

Also, the present disclosure provides a recombinant vector comprising at least one selected from the group consisting of: the nucleotide sequence of Nanovirus DNA-C represented by SEQ ID NO: 5; the nucleotide sequence of Nanovirus DNA-M represented by SEQ ID NO: 6; the nucleotide sequence of Nanovirus DNA-N represented by SEQ ID NO: 7; and the nucleotide sequence of Nanovirus DNA-U1 represented by SEQ ID NO: 8.

As used herein, the term “Nanovirus ” refers to a genus of plant viruses in the family Nanoviridae, with single-stranded circular DNAs as the genome thereof. There are eight species in this genus. Of them, seven species have eight genome components while the remaining one has six genome components. The genome components are typically around 1 kb in length with one ORF per component.

In an embodiment of the present disclosure, examples of the Nanovirus include MDV (Milk Vetch dwarf virus), FBNYV (Faba bean necrotic yellows virus), FBNSV (Faba bean necrotic stunt virus), SCSV (Subterranean clover stunt virus), PNYDV (Pea necrotic yellow dwarf virus), BBTV (Banana bunchy top virus), MVCDV (Milk vetch chlorotic dwarf virus), CBDV (Cardamom bushy dwarf virus), ABTV (Abaca bunchy top virus), and CvLV (Cow vetch latent virus), but are not limited thereto.

Specifically, the Nanovirus according to an embodiment of the present disclosure may be at least one selected from the group consisting of MDV (Milk Vetch dwarf virus), FBNYV (Faba bean necrotic yellows virus), FBNSV (Faba bean necrotic stunt virus, FBNSV), SCSV (Subterranean clover stunt virus), PNYDV (Pea necrotic yellow dwarf virus), BBTV (Banana bunchy top virus), MVCDV (Milk vetch chlorotic dwarf virus), CBDV (Cardamom bushy dwarf virus), ABTV (Abaca bunchy top virus), and CvLV (Cow vetch latent virus).

The said term “Faba bean necrotic stunt virus (FBNSV)” refers to a multipartite virus belonging to the Nanovirus genus in the Nanoviridae family. FBNSV has eight circular single-stranded DNA components, each approximately 1 kb in length. Additionally, two alphasatellites are commonly associated with FBNSV infection. The only full-length genome sequence of an Iranian FBNSV isolate, FBNSV, infecting faba bean, has been reported. To explore the diversity of Iranian FBNSV isolates (FBNSV-IRs), researchers characterized the full-length genomes of four FBNSV isolates and associated alphasatellites obtained from faba bean, common bean, and chickpea. All genomic components, except U4, matched the expected sizes based on GenBank sequences. U4 displayed an additional 16-nucleotide stretch in its non-coding region. The alphasatellites from these isolates exhibited 98-100% sequence identity. Intra-component sequence analyses of FBNSV-IRs indicated high nucleotide identity (98-100%) between individual components or their associated alphasatellites, with DNA-M and DNA-U2 showing the highest diversity, sharing 98-99% identity.

The said term “Subterranean clover stunt virus (SCSV)” is a single-stranded DNA virus that can infect legumes and subterranean clover is the main host plant. As the type of member of the Nanovirus genus, SCSV is characterized by a multipartite genome consisting of eight components, each around 1 kb in size. Aphids transmit SCSV in a non-propagative circulative manner, replicating within the aphid but not passing on to its offspring. Infection by SCSV results in severe stunting, chlorosis, and deformation of the host plants, causing significant yield losses in pastures and legume crops. This virus poses a global threat to subterranean clover cultivation, with symptoms including stunting and yellowing, leading to reduced plant growth and forage quality. Insect vectors like aphids play a critical role on its widespread transmission of SCSV in agriculture fields including its genomic organization and pathogenic mechanisms, is imperative for developing effective management strategies to mitigate its impact on subterranean clover crops and, consequently, ensuring the sustainability of agriculture.

The said term “Peanecrotic yellow dwarf virus (PNVDV)” is a plant infectious virus, initially identified in pea crops in Saxony-Anhalt, Germany in 2009, and was later spread countrywide in both Germany and Austria by 2016, affecting not only peas but also faba bean, vetch, and lentil, resulting in significant yield losses. PNYDV is transmitted by aphids in a circulative, non-propagative manner, and the potential for increased Nanovirus infection is anticipated due to changing climatic conditions, particularly milder winters in Central Europe, which favor aphid survival and virus spread vastly. The virus was first detected in The Netherlands in 2017, indicating the widespread presence of nanoviruses across Europe. The increasing geographic range of PNYDV highlights the need for vigilance and proactive measures to address potential threats to leguminous crops in the region.

The said term “Banana Bunchy Top Virus (BBTV)” refers to the Nanovirus that belongs to the genus Babuvirus in the family Nanoviridae and is a significant threat to banana cultivation causing Banana Bunchy Top Disease (BBTD). The disease was first reported in the Fiji Islands in 1889, BBTV's causal agent was identified nearly a century later. BBTD has been reported in approximately 33 countries across Africa, Asia, Australia, and the South Pacific Islands but not in the Americas. The virus is transmitted by the banana aphid (Pentalonia nigronervosa) in a persistent circulative and non-replicative manner. Unlike aphid-transmitted luteoviruses, BBTV's transmission mechanism differs. The banana aphid, highly specific to Musa spp., has a global presence in banana-growing countries. BBTV primarily spreads through infected vegetative propagules, and it's not mechanically transmissible or facilitated by contact with agricultural materials. Although BBTD is prevalent in Asia and the South Pacific, it is not uniformly distributed across all banana-growing regions, and notably absent in Central and South America and the Caribbean. Despite BBTV isolations in Australia, Hawaii, Indonesia, Tonga, and Taiwan, the association of BBTV with the disease remains unconfirmed in many affected countries.

The said term “Milk vetch chlorotic dwarf virus (MVCDV)” refers to a novel Nanovirus, which was discovered in Iran infecting milk vetch plants (Astragalus myriacanthus Boiss.; family Fabaceae) showing symptoms like marginal leaf chlorosis, little leaves, and dwarfing. Using conventional PCR and high-throughput sequencing, all eight segments (DNA-C, DNA-M, DNA-N, DNA-R, DNA-S, DNA-U1, DNA-U2, and DNAU4) were identified and sequenced, leading to the characterization of a new Nanovirus named MVCDV. The MVCDV genome shares 62.2-74.7% nucleotide pairwise identity with other nanoviruses, with DNA-C, DNA-M, DNA-N, and DNA-S components being closely related to those of Black Medic Leaf Roll Virus (BMLRV). Additionally, three nanoalphasatellites (family Alphasatellitidae) associated with MVCDV were identified, belonging to different genera. Given the diversity of Astragalus spp. In Iran, it is suggested that more nanoviruses may exist in these plants, potentially influencing the spread of nanoviruses to cultivated fabaceous hosts.

The said term “Cardamom bushy dwarf virus (CBDV)” represents the first distinct Babuvirus species infecting a plant outside the Musaceae family, showing additional diversity within the Babuvirus genus. In India in 1936, CBDV is associated with causing foorkey disease that poses a significant threat to large cardamom production, characterized by the extensive proliferation of stunted shoots rendering the plant unproductive. In research it is found that the incidence (37.2-39.3%) of foorkey was observed in certain plantations in the Darjeeling hills, and CBDV was detected in infected plants by PCR. The comprehensive analysis revealed nine novel DNA components in CBDV, including major components (DNA-R, -S, -M, -C, -N, and -U3) resembling those found in the genus Babuvirus within the family Nanoviridae. Additional components, satellite Rep (DNA-sRep1), and unknown components (DNA-Uf1 and -Uf2) were also identified, with genome component sizes ranging from 1028 to 1127. CBDV shows the different sequence identity and phylogeny from the Babuvirus species, such as Banana Bunchy Top Virus and Abaca Bunchy Top Virus.

The said term “Abaca bunchy top virus (ABTV)” refers to genus Babuvirus, which is classified within the Nanoviridae family. This virus is transmitted by the banana aphid Pentalonia nigronervosa, persistently but non-propagatively, through vegetative plant material. ABTV has multi-component genomes, each independently encapsidated in a single virion. Despite sharing 79-81% amino acid sequence identity in their coat proteins and 54-76% nucleotide sequence identity across all component genomes, ABTV has distinct species within the same genus. These viruses can occur in single or mixed infections in abaca, often without showing early symptoms, posing challenges for disease control strategies. Early detection of Abaca bunchy top disease (ABTD) is crucial for timely removal of diseased plants and ensuring planting materials are free of ABTV. Detection methods for bunchy top viruses are largely derived from BBTV nucleic acid-based and serological assays applied in banana samples. ABTD is economically significant in the Philippines, causing bunched top appearance, stunting, yellowing of leaves, poor fiber quality, and substantial fiber yield loss. The symptoms and transmission manner of ABTD are like Banana Bunchy Top Disease (BBTD) in bananas, and ABTD has been reported in the Philippines since 1910.

The said term “Cow vetch latent virus (CvLV)” refers to a virus, which consists of eight single-stranded DNA components, each approximately 1 kb in size and names of genomic components based on the functions of the respective encoded proteins. These components include DNA-R (master replication initiator protein), DNA-S (structural capsid protein), DNA-C (cell cycle link protein), DNA-M (movement protein), and DNA-N (nuclear shuttle protein). Three other DNA molecules, DNA-U1, DNA-U2, and DNA-U4, are typical constituents of a Nanovirus genome, although the functions of the proteins they encode are not yet known. CvLV is the second Nanovirus reported from a wild perennial host after Sophora Yellow. The presence of Stunt-Associated Virus in Sophora alopecuroides plants in Iran underscores the significance of exploring virus diversity across both cultivated and uncultivated host species. This examination reveals that nanoviruses within the Nanoviridae family might have a wider environmental distribution than current knowledge of known species implies. As efforts concentrate on uncultivated hosts, the anticipation of discovering new nanoviruses heightens, promising valuable insights into the ecological and evolutionary origins of these viruses responsible for diseases in cultivated hosts. Moreover, CvLV features two alphasatellites exhibiting a 64% pairwise identity between themselves and less than an 81% identity with other established Nanovirus -associated alphasatellites. This disparity suggests a distinctive genetic relationship within the Nanovirus family.

More specifically, the Nanovirus of the present disclosure may be MDV (Milk Vetch dwarf virus) and/or FBNYV (Faba bean necrotic yellows virus).

As used herein, the term “Milk vetch dwarf virus” (MDV) refers to a DNA virus of the genus Nanovirus in the family Nanoviridae, and the virus primarily infects a range of leguminous species, causing dwarfism, chlorosis, and wrinkles.

As used herein, the term “Faba bean necrotic yellows virus” (FBNYV) refers to a DNA virus of the genus Nanovirus in the family Nanoviridae. It primarily infects a range of leguminous species, causing dwarfism, leaf curling, and yellowing.

Particularly, the Nanovirus (e.g., Milk vetch dwarf virus (MDV) and Faba bean necrotic yellows virus (FBNYV)) has a multipartite genomic organization composed of at least eight genomes including four separated genomes DNA-C, DNA-M, DNA-N, and DNA-U1. It is known that normal viral replication and symptom expression are induced only when all eight genomes are present. DNA-C codes for a cell-cycle link protein, DNA-M for a movement protein, and DNA-N for a nuclear shuttle protein. It has not yet been revealed exactly what ‘function performed by protein of DNA-U1.

In addition to the genome segments, the Nanovirus possesses two more separated genomes DNA-R and DNA-S which encode a replication initiator protein (Rep) and a coat protein, respectively.

Variants of the nucleotide sequences carried by the recombinant vector fall within the scope of the present disclosure. It should be appreciated that the nucleotide sequences of SEQ ID NOS: 1 to 8 of the present disclosure include functional equivalents of the constituent nucleic acid molecules, that is, variants which show the same functions as those of the intact nucleic acid molecules although they are mutated by deletion, substitution, or insertion of some nucleotide residues. Specifically, the gene may contain a nucleotide sequence with at least 70%, preferably at least 80%, more preferably at least 90%, and most preferably at least 95% sequence homology with the nucleotide sequences of SEQ ID NOS: 1 to 8.

The recombinant vector of the present disclosure may carry genes of Nanovirus DNA-C, DNA-M, DNA-N, and DNA-U1. The genes of Nanovirus DNA-C, DNA-M, DNA-N, and DNA-U1 are subcloned into a dimer in the vector. In this regard, two monomers of each component are ligated by using different restriction enzymes and linked within a plasmid.

As used herein, the term “vector” refers to a carrier for expressing a gene of interest in a host cell and is intended to encompass plasmid vectors; E. coli vectors; Agrobacterium vectors; and bacterial or viral vectors such as bacteriophage vectors, adenovirus vectors, retrovirus vectors, and adeno-associated virus vectors, with preference for E. coli vectors, but it is not limited thereto.

In an embodiment of the present disclosure, the transformed E. coli and Agrobacterium were prepared using the recombinant vector. It was observed that when these transformed Agrobacterium strains harboring Nanovirus genes infected plants, these agroinoculated plants showed the specific disease symptoms shown Nanovirus infected plants in nature. Therefore, the present disclosure provides Agrobacterium tumefaciens comprising the recombinant vector.

As used herein, the term “Agrobacterium” refers to a motile soil microbe that lives in the soil and carries a tumor-inducing (Ti) plasmid. It infects plants through wounds, causing abnormal proliferation of tissue cells.

In an embodiment of the present disclosure, Agrobacterium capable of expressing Nanovirus DNA-C, DNA-M, DNA-N, or DNA-U1 were developed by transformation with the recombinant vector.

Specifically, an embodiment of the present disclosure may employ Nanovirus DNA-C-expressing Agrobacterium tumefaciens expressing, which was deposited with the Korean Agricultural Culture Collection (KACC) under the accession number KACC 92475 on Nov. 22, 2022.

Another embodiment of the present disclosure may employ Nanovirus DNA-M-expressing Agrobacterium tumefaciens, which was deposited with the Korean Agricultural Culture Collection (KACC) under the accession number KACC 92476 on Nov. 22, 2022.

Another particular embodiment of the present disclosure may employ Nanovirus DNA-N-expressing Agrobacterium tumefaciens, which was deposited with the Korean Agricultural Culture Collection (KACC) under the accession number KACC 92477 on Nov. 22, 2022.

Another particular embodiment of the present disclosure may employ Nanovirus DNA-U1-expressing Agrobacterium tumefaciens, which was deposited with the Korean Agricultural Culture Collection (KACC) under the accession number KACC 92478 on Nov. 22, 2022.

Another particular embodiment of the present disclosure may employ Nanovirus DNA-C-expressing Agrobacterium tumefaciens, which was deposited with the Korean Agricultural Culture Collection (KACC) under the accession number KACC 92479 on Nov. 22, 2022.

Another particular embodiment of the present disclosure may employ Nanovirus DNA-M-expressing Agrobacterium tumefaciens, which was deposited with the Korean Agricultural Culture Collection (KACC) under the accession number KACC 92480 on Nov. 22, 2022.

Another particular embodiment of the present disclosure may employ Nanovirus DNA-N-expressing Agrobacterium tumefaciens, which was deposited with the Korean Agricultural Culture Collection (KACC) under the accession number KACC 92481 on Nov. 22, 2022.

Another particular embodiment of the present disclosure may employ Nanovirus DNA-U1-expressing Agrobacterium tumefaciens, which was deposited with the Korean Agricultural Culture Collection (KACC) under the accession number KACC 92482 on Nov. 22, 2022.

The term “transformation”, as used herein, means that a foreign DNA or RNA is absorbed into cells to change the genotype of the cells. In this regard, the host cells include plant cells, prokaryotic cells, yeast cells, or insect cells, but are not limited to them.

Another aspect of the present disclosure provides a Nanovirus -infected plant transformed with the recombinant vector.

Specifically, the present disclosure provides a Nanovirus -infected plant transformed with a vector comprising at least one selected from the group consisting of: the nucleotide sequence of Nanovirus DNA-C, represented by SEQ ID NO: 1; the nucleotide sequence of Nanovirus DNA-M, represented by SEQ ID NO: 2; the nucleotide sequence of Nanovirus DNA-N, represented by SEQ ID NO: 3; and the nucleotide sequence of Nanovirus DNA-U1, represented by SEQ ID NO: 4.

Also, the present disclosure provides a Nanovirus -infected plant transformed with a vector comprising at least one selected from the group consisting of: the nucleotide sequence of Nanovirus DNA-C, represented by SEQ ID NO: 5; the nucleotide sequence of Nanovirus DNA-M, represented by SEQ ID NO: 6; the nucleotide sequence of Nanovirus DNA-N, represented by SEQ ID NO: 7; and the nucleotide sequence of Nanovirus DNA-U1, represented by SEQ ID NO: 8.

As described above, the Nanovirus (e.g., Milk vetch dwarf virus (MDV) and Faba bean necrotic yellows virus (FBNYV)) has a multipartite genomic organization composed of at least eight genomes. It is known that normal viral replication and symptom expression are induced only when all eight genomes are present.

In the present disclosure, a Nanovirus -infected plant is created by additional transformation with Agrobacterium tumefaciens containing a nucleotide sequence of Nanovirus DNA-R coding for the Rep protein associated with DNA replication and a nucleotide sequence of DNA-S coding for a coat protein in addition to transformation with Nanovirus DNA-C, DNA-M, DNA-N, and DNA-U1.

Therefore, in an embodiment of the present disclosure, the Nanovirus -infected plant may be additionally transformed with a recombinant vector comprising the nucleotide sequence of Nanovirus DNA-R, represented by SEQ ID NO: 9, the nucleotide sequence of Nanovirus DNA-S, represented by SEQ ID NO: 10, or a combination thereof.

In accordance with another embodiment of the present disclosure, the Nanovirus -infected plant may be additionally transformed with a recombinant vector comprising the nucleotide sequence of Nanovirus DNA-R, represented by SEQ ID NO: 11, the nucleotide sequence of Nanovirus DNA-S, represented by SEQ ID NO: 12, or a combination thereof.

Target plants in the present disclosure may include legumes such as faba beans, lentils, and peas, but no limitations are imparted to the plants that are susceptible to infection by any Nanovirus DNA-C, -M, -N, and -U1. Specifically, the target plants of the present disclosure may include those susceptible to infection by Milk vetch dwarf virus (MDV) and/or Faba bean necrotic yellows virus (FBNYV) DNA-C, -M, -N, and -U1.

Additionally, the plant cell into which the recombinant vector of the present disclosure is introduced is not particularly limited to a specific form if the cell can be regenerated into a plant. These cells may include, for example, cultured cell suspensions, protoplasts, leaf sections, or calluses.

The introduction of the recombinant vector into plants may rely on methods for inoculation well known for in the art, examples of which include, but are not limited to, agrobacterium-mediated methods, particle gun bombardment, silicon carbide whiskers, sonication, heat shock, electroporation, and PEG (polyethylene glycol) precipitation.

Specifically, Agrobacterium tumefaciens may be used to induce Nanovirus -specific disease symptoms, caused by Nanovirus DNA-C, DNA-M, DNA-N, and DNA-U1, into plants. As used herein, the Agro-inoculation method refers to a technique for introducing viral genes into plants for expressing or producing desired proteins within the plants. In the Agro-inoculation, a wound is created at the apex of the plant intended for inoculation using an insect pin and then a suspension culture of Agrobacterium containing the Nanovirus infectious clone and activation buffer is injected through the wound.

Thus, contemplated according to another aspect of the present disclosure is a method for inducing Nanovirus disease in plants, the method including a step of transfecting the recombinant vector into the plant.

Specifically, the present disclosure provides a method for inducing Nanovirus disease in plants, the method including a step of transfecting a vector into the plant comprising at least one Nanovirus segment from the group consisting of the nucleotide sequence of Nanovirus DNA-C, represented by SEQ ID NO: 1; the sequence of Nanovirus DNA-M, , represented by SEQ ID NO 2; the nucleotide sequence of Nanovirus DNA-N, represented by SEQ ID NO 3; and the nucleotide sequence of Nanovirus DNA-U1, represented by SEQ ID NO 4.

In addition, specifically, the present disclosure provides a method for inducing a Nanovirus disease symptom in a plant, the method including a step of transfecting into the plant a recombinant vector comprising at least one Nanovirus segment selected from the group consisting of: the nucleotide sequence of Nanovirus DNA-C, represented by SEQ ID NO: 5; the nucleotide sequence of Nanovirus DNA-m, represented by SEQ ID NO: 6; the nucleotide sequence of Nanovirus DNA-N, represented by SEQ ID NO: 7; and the nucleotide sequence of Nanovirus DNA-U1, represented by SEQ ID NO: 8.

More specifically, according to the present disclosure, a Nanovirus -infected plant expressing a Nanovirus -specific disease symptom can be obtained by transfecting the Agrobacterium tumefaciens comprising a recombinant vector comprising at least one Nanovirus segment selected from the group consisting of: the nucleotide sequence of Nanovirus DNA-C, represented by SEQ ID NO: 1; the nucleotide sequence of Nanovirus DNA-M, represented by SEQ ID NO: 2; the nucleotide sequence of Nanovirus DNA-N, represented by SEQ ID NO: 3; and the nucleotide sequence of Nanovirus DNA-U1, represented by SEQ ID NO: 4 into the plant.

More specifically, according to the present disclosure, a Nanovirus -infected plant expressing a Nanovirus -specific disease symptom can be obtained by transfecting into the plant the Agrobacterium tumefaciens comprising a recombinant vector comprising at least one selected from the group consisting of: the nucleotide sequence of Nanovirus DNA-C, represented by SEQ ID NO: 5; the nucleotide sequence of Nanovirus DNA-M, represented by SEQ ID NO: 6; the nucleotide sequence of Nanovirus DNA-N, represented by SEQ ID NO: 7; and the nucleotide sequence of Nanovirus DNA-U1, represented by SEQ ID NO: 8.

In an embodiment of the present disclosure, the method may further include a step of transfecting into the plant a recombinant vector comprising the nucleotide sequence of Nanovirus DNA-R, represented by SEQ ID NO: 9, a recombinant vector comprising the nucleotide sequence of Nanovirus DNA-S, represented by SEQ ID NO: 10, or a combination thereof at the step of transfecting into a plant.

In another embodiment of the present disclosure, the method may further include a step of transfecting into the plant a recombinant vector comprising the nucleotide sequence of Nanovirus DNA-R, represented by SEQ ID NO: 11, a recombinant vector comprising the nucleotide sequence of Nanovirus DNA-S, represented by SEQ ID NO: 12, or a combination thereof at the step of transfecting into a plant.

Provided according to another aspect of the present disclosure is a composition for the diagnosis of Nanovirus infection in plant, the composition includes:
(a) an agent for detecting Nanovirus DNA-C including the nucleotide sequence of SEQ ID NO: 1 or a fragment thereof, Nanovirus DNA-M including the nucleotide sequence of SEQ ID NO: 2 or a fragment thereof, Nanovirus DNA-N including the nucleotide sequence of SEQ ID NO: 3 or a fragment thereof, Nanovirus DNA-U1 including the nucleotide sequence of SEQ ID NO: 4 or a fragment thereof, or a combination thereof.
(b) an agent for detecting Nanovirus DNA-C including the nucleotide sequence of SEQ ID NO: 5 or a fragment thereof, Nanovirus DNA-M including the nucleotide sequence of SEQ ID NO: 6 or a fragment thereof, Nanovirus DNA-N including the nucleotide sequence of SEQ ID NO: 7 or a fragment thereof, Nanovirus DNA-U1 including the nucleotide sequence of SEQ ID NO: 8 or a fragment thereof, or a combination of them.

Hence, the composition for the diagnosis of Nanovirus infection can be used to determine whether a plant is infected with Norovirus or not.

In an embodiment of the present disclosure, the composition for the diagnosis of Nanovirus infection may further include:
(a′) an agent for detecting DNA-R including the nucleotide sequence of SEQ ID NO: 9 or a fragment thereof, DNA-S including the nucleotide sequence of SEQ ID NO: 10 or a fragment thereof, or a combination thereof.
(b′) an agent for detecting DNA-R including the nucleotide sequence of SEQ ID NO: 11 or a fragment thereof, DNA-S including the nucleotide sequence of SEQ ID NO: 12 or a fragment thereof, or a combination thereof; or
(c′) a combination thereof.

As used herein, the term “composition” refers to a collection of reagents used for DNA detection and amplification, including primers for detecting the causative agents from the symptomatic infected plants, DNA polymerase, dNTPs, and buffer.

As used herein, the term “diagnosis” means determining the presence or absence of an infection by amplifying the genes of an individual segment showing specific disease symptoms after infection using the primer set of the present disclosure.

In an embodiment of the present disclosure, the agent for detecting the virus further includes primers or a probe that specifically bind to Nanovirus DNA-C, DNA-M, DNA-N, or DNA-U1.

Furthermore, in another embodiment of the present disclosure, the agent for detecting the virus further includes primers or a probe that specifically bind to Nanovirus DNA-R or DNA-S.

Specifically, the primers or a probe may specifically bind to DNA-C, DNA-M, DNA-N, or DNA-U1 of Milk vetch dwarf virus (MDV) and/or Faba bean necrotic yellows virus (FBNYV), which belong to the genus Nanovirus.

The term “specifically binding” pertains to a nucleotide sequence capable of synthesizing a PCR product using a target gene of diagnosis as a template, only when the target gene is present, without synthesizing PCR products in response to other genes.

The composition may further include a reaction amplification mixture. The mixture refers to the reagents necessary for carrying out the amplification reaction, including a thermostable DNA polymerase, deoxynucleotides, nuclease-free sterile water, and a solution containing divalent metal cations. Preferably, the mixture may include reaction buffer, deoxynucleotides, and DNA polymerase.

Additionally, the probe may have a reporter, such as a fluorescent material, labeled to the end thereof.

As used herein, the term “primer” refers to a short single-stranded nucleic acid sequence having a free 3′ hydroxyl group, which can undergo base-pairing interaction with a complementary template and serves as a starting point for replicating the desired template strand. A primer can initiate DNA synthesis in the presence of a reagent for polymerization (e.g., DNA polymerase or reverse transcriptase) and four different nucleoside triphosphates in suitable buffers and at a suitable temperature.

When designing the primers, there are various restrictions, such as the A, G, C, and T content ratio of the primers, prevention of primer complex (dimer) formation, and prohibition of repeating the same base sequence more than three times. In addition, individual PCR reactions need appropriate conditions, such as the amount of template DNA, concentration of primers, concentration of dNTP, concentration of Mg²⁺, reaction temperature, reaction time, etc.

The primer set according to the present disclosure may include additional features that do not change the basic properties. That is, the nucleic acid sequence can be modified by using different methods. Examples of such modifications may include methylation, capping, substitution of one or more homologs of a nucleotide, and modification of a nucleotide with an uncharged linkage such as phosphonate, phosphodiester, phosphonamidite, or carbamate, or charged linkage such as phosphonothioate or phosphorodithioate. Further, the nucleic acid may have one or more residues, which are additionally covalent-bonded, such as nuclease, a toxin, an antibody, a signal peptide, a poly-L-lysine, an intercalating agent such as acridine and psoralen, a chelating agent such as a metal, a radioactive metal, and an iron-oxidizing metal, and alkylation agent.

Further, the nucleotide sequence of the primer set according to the present disclosure can be modified using a label capable of directly or indirectly providing a detectable signal. The primer and primer set may include a label that can be detected using spectroscopic, photochemical, biochemical, immunochemical, or chemical means. Useful labels include ³²P, fluorescent dyes, electron dense reagents, enzymes (commonly used in ELISA), biotin or hapten, and proteins that are available for antisera or monoclonal antibodies.

The primer set according to the present disclosure can be chemically synthesized using any other well-known method including a phosphotriester method such as cloning and restriction enzyme digestion of appropriate sequences and Narang (1979, Meth, Enzymol. 68: 90-99), a diethylphosphoramidate method such as Beaucage (1981, Tetrahedron Lett. 22: 1859-1862), and the direct chemical synthesis methods such as the solid support methods of U.S. Pat. No. 4,458,066.

Another aspect of the present disclosure provides a method for diagnosing Nanovirus infection in a plant, using the composition of the diagnosis of Nanovirus infection.

Specifically, the present disclosure provides a method for diagnosing Nanovirus infection in a plant, the method including the steps of composition for the detection of Nanovirus infection with a plant-derived DNA extraction and performing a nucleic acid amplification reaction; and detecting the amplified nucleic acid by specific primers.

In an embodiment of the present disclosure, the amplified nucleic acid may be selected from:
(aa) Nanovirus DNA-C including the nucleotide sequence of SEQ ID NO: 1 or a fragment thereof, Nanovirus DNA-M including the nucleotide sequence of SEQ ID NO: 2 or a fragment thereof, Nanovirus DNA-N including the nucleotide sequence of SEQ ID NO: 3 or a fragment thereof, Nanovirus DNA-U1 including the nucleotide sequence of SEQ ID NO: 4 or a fragment thereof, or a combination thereof.
(bb) Nanovirus DNA-C including the nucleotide sequence of SEQ ID NO: 5 or a fragment thereof, Nanovirus DNA-M including the nucleotide sequence of SEQ ID NO: 6 or a fragment thereof, Nanovirus DNA-N including the nucleotide sequence of SEQ ID NO: 7 or a fragment thereof, Nanovirus DNA-U1 including the nucleotide sequence of SEQ ID NO: 8 or a fragment thereof, or a combination thereof; or
(cc) a combination thereof.

Specifically, the method for diagnosing Nanovirus infection according to the present disclosure means a method for determining whether a suspected plant is infected by virus or not by identifying the presence or absence of Nanovirus infection in the plant through a nucleic acid amplification reaction using the composition for the diagnosis of Nanovirus infection.

More specifically, the method for diagnosing Nanovirus infection according to an embodiment of the present disclosure, includes the steps of: contacting the composition for the diagnosis of Nanovirus infection with a plant-derived sample DNA and performing a nucleic acid amplification reaction; and detecting Nanovirus DNA-C including the nucleotide sequence of SEQ ID NO: 1 or a fragment thereof, Nanovirus DNA-M including the nucleotide sequence of SEQ ID NO: 2 or a fragment thereof, Nanovirus DNA-N including the nucleotide sequence of SEQ ID NO: 3 or a fragment thereof, Nanovirus DNA-U1 including the nucleotide sequence of SEQ ID NO: 4 or a fragment thereof, or a combination thereof, whereby the presence of a specific gene of Nanovirus in the plant is identified.

In addition, more specifically, the method for diagnosing Nanovirus infection according to another embodiment of the present disclosure includes the steps of: contacting the composition for the diagnosis of Nanovirus infection with a plant-derived DNA sample and performing a nucleic acid amplification reaction; and detecting Nanovirus DNA-C including the nucleotide sequence of SEQ ID NO: 5 or a fragment thereof, Nanovirus DNA-M including the nucleotide sequence of SEQ ID NO: 6 or a fragment thereof, Nanovirus DNA-N including the nucleotide sequence of SEQ ID NO: 7 or a fragment thereof, Nanovirus DNA-U1 including the nucleotide sequence of SEQ ID NO: 8 or a fragment thereof, or a combination thereof, whereby the presence of a specific gene of Nanovirus in the plant is identified.

Also, in an embodiment of the present disclosure, the amplified nucleic acid may further include:
(aa′) Nanovirus DNA-R including the nucleotide sequence of SEQ ID NO: 9 or a fragment thereof, Nanovirus DNA-S including the nucleotide sequence of SEQ ID NO: 10 or a fragment thereof, or a combination thereof.
(bb′) Nanovirus DNA-R including the nucleotide sequence of SEQ ID NO: 11 or a fragment thereof, Nanovirus DNA-S including the nucleotide sequence of SEQ ID NO: 12 or a fragment thereof, or a combination thereof; or
(cc′) a combination thereof.

As used herein, the term “amplification reaction” refers to a polymerase chain reaction (PCR) for amplifying a nucleic acid molecule, examples of which include reverse transcription polymerase chain reaction (RT-PCR), ligase chain reaction (LCR), Gap-LCR (WO 90/01069), repair chain reaction (EP 439,182), transcription-mediated amplification (TMA) (WO88/10315), self-sustained sequence replication (WO90/06995), selective amplification of target polynucleotide sequences, consensus sequence primed polymerase chain reaction (CP-PCR), arbitrarily primed polymerase chain reaction (AP-PCR), nucleic acid sequence based amplification (NASBA), strand displacement amplification, and loop-mediated isothermal amplification (LAMP), but are not limited thereto.

Since the method for diagnosing Nanovirus infection of the present disclosure includes the combination for the diagnosis of Nanovirus infection according to another aspect of the present disclosure, the redundant content is incorporated by reference and the description thereof is omitted to avoid undue complexity of the specification.

When used, a recombinant vector comprising the nucleotide sequences of Nanovirus DNA-C, DNA-M, DNA-N, and DNA-U1 represented by SEQ ID NOS: 1 to 8, and E. coli and Agrobacterium tumefaciens, each transformed therewith, can effectively induce the infection of Nanovirus into crops even without any insect vectors.

Therefore, the present disclosure can be applied to various studies, such as investigating the host range and the correlation with hosts of Nanovirus DNA-C, DNA-M, DNA-N, and DNA-U1. This can help in preemptively preventing economic losses due to virus infections, making it beneficially applicable in the related fields.

### BRIEF DESCRIPTION OF THE DRAWINGS

FIG. 1 is a schematic view illustrating processes of constructing a recombinant vector available as an infectious clone of milk vetch dwarf virus (MDV) DNA-C.

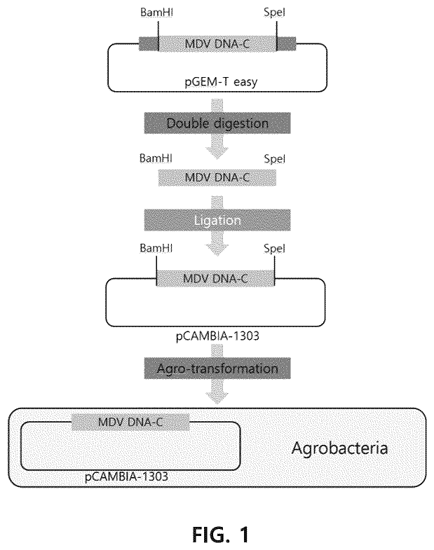

FIG. 2 is a schematic view illustrating processes of constructing a recombinant vector available as an infectious clone of MDV DNA-M.

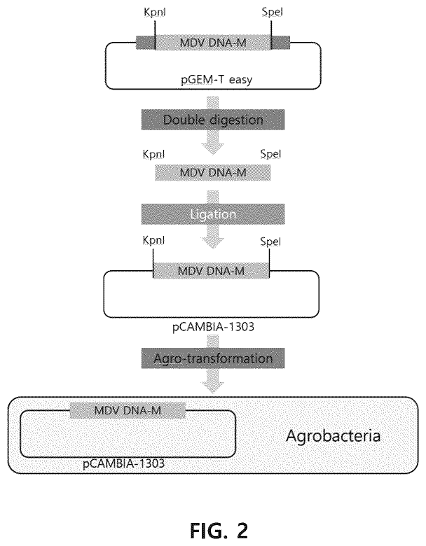

FIG. 3 is a schematic view illustrating processes of constructing a recombinant vector available as an infectious clone of MDV DNA-N;

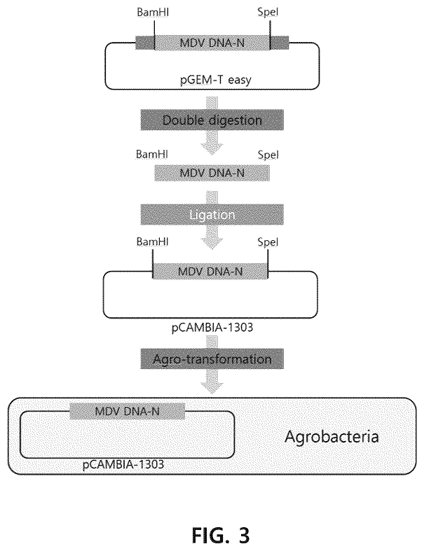

FIG. 4 is a schematic view illustrating processes of constructing a recombinant vector available as an infectious clone of MDV DNA-U1;

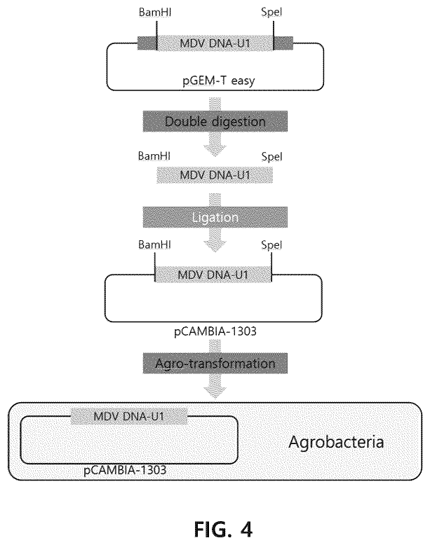

FIG. 5 is a schematic view illustrating processes of constructing a recombinant vector available as an infectious clone of faba bean necrotic yellows virus (FBNYV) DNA-C;

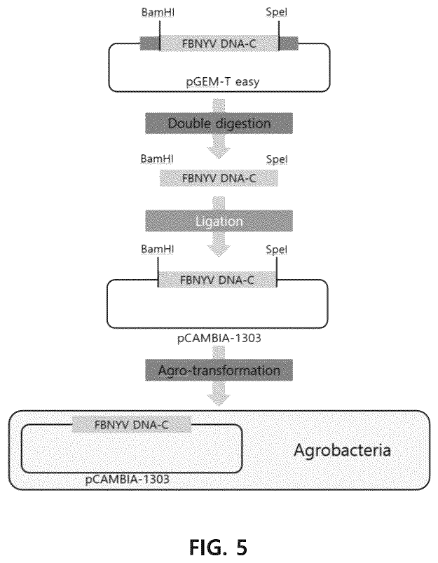

FIG. 6 is a schematic view illustrating processes of constructing a recombinant vector available as an infectious clone of FBNYV DNA-M;

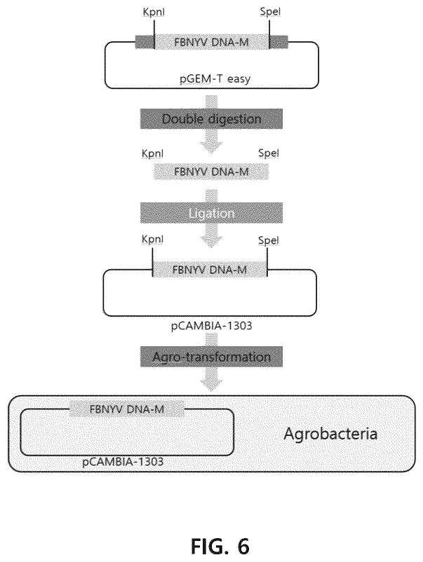

FIG. 7 is a schematic view illustrating processes of constructing a recombinant vector available as an infectious clone of FBNYV DNA-N;

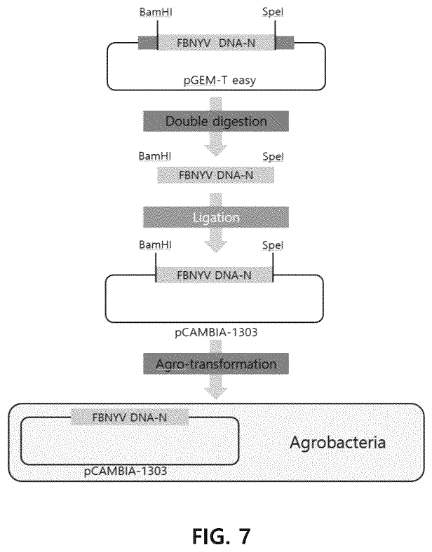

FIG. 8 is a schematic view illustrating processes of constructing a recombinant vector available as an infectious clone of FBNYV DNA-U1; and

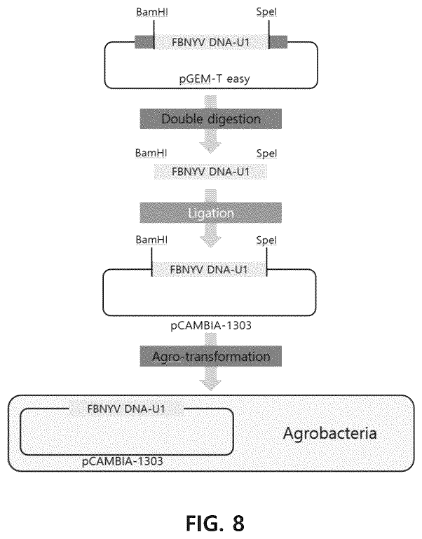

FIG. 9 shows results of identifying the infection of Nanovirus in host plants: (a) infection of milk vetch dwarf virus (MDV) in host plants by infectious clones of MDV DNA-C, DNA-M, DNA-N, and DNA-U1, and (b) infection of faba bean necrotic yellows virus (FBNYV) in host plants by infectious clones of FBNYV DNA-C, DNA-M, DNA-N, and DNA-U1.

### DETAILED DESCRIPTION OF THE EXEMPLARY EMBODIMENTS

Hereinafter, the present disclosure will be described in more detail through examples. These examples are only for illustrating the present disclosure in more detail, and it will be apparent to those skilled in the art that the scope of the present disclosure is not limited by these examples according to the gist of the present disclosure.

### EXAMPLES

Unless otherwise stated, “%” used to indicate the concentration of a specific substance is (weight/weight) % for solid/solid, (weight/volume) % for solid/liquid, and (volume/volume) % for liquid/liquid throughout the specification.

### Example 1. Acquisition of DNA-C, DNA-M, DNA-N, and DNA-U1 Genes of MDV and FBNYV

### EXAMPLE 1.1. DNA-C, -M,-N, and -U1 Genes of Milk Vetch Dwarf Virus (MDV)

MDV DNA was obtained from papaya samples infected with milk vetch dwarf virus (MDV) in a farm in Yesan, South Chungcheong Province. Rolling circle amplification was performed using the TempliPhi™ 100 Amplification Kit from GE Healthcare to specifically amplify only circular DNA from the DNA. The amplified DNA was then treated with restriction enzymes EcoRI and PstI, and electrophoresed on an agarose gel to isolate DNA fragments approximately 1 kb in length, presumed to be from Nanovirus. The isolated DNA was ligated into pCAMBIA 1303 using T4 DNA ligase and transformed into Escherichia coli DH5α strain which was then cultured at 37° C. Plasmids were extracted from DH5α using AccuPrep® Nano-Plus Plasmid Mini Extraction Kit from Bioneer Inc. and sent to Macrogen Inc. for sequencing analysis to determine the nucleotide sequences. These sequences have been registered in the National Center for Biotechnology Information (NCBI) GenBank (KY070230, MN795302, LC094150, and KY070231).

### Example 1.2. DNA-C, -M, -N, and -U1 Genes of Faba Bean Necrotic Yellows Virus (FBNYV)

Based on the FBNYV DNA-C, DNA-M, DNA-N, and DNA-U1 sequences (MG950210, MG950211, MG950212, and MG950213) registered in the National Center for Biotechnology Information (NCBI) GenBank from Iran, the genes were rearranged so that non-coding regions, which were not part of the gene coding portion, but included intergenic regions, were repeated before and after the coding sequence. The resulting sequences were synthesized through the gene synthesis service of Genewiz (SEQ ID NOS: 1 to 4). For cloning purposes, restriction enzyme recognition sites were added to the front and back of the sequences. Specifically, BamHI (GGATCC) and SpeI (ACTAGT) were added to DNA-C, KpnI (GGTACC) and SpeI (ACTAGT) to DNA-M, BamHI (GGATCC) and SpeI (ACTAGT) to DNA-N, and BamHI (GGATCC) and SpeI (ACTAGT) to DNA-U1.

### Example 2. Construction of Infectious Clones of MDV and FBNYV DNA-C, DNA-M, DNA-N, and DNA-U1

First, the genes synthesized in Example 1 were cloned into a pGEM T-easy vector and amplified in DH5α, and the plasmids were extracted. (i) For DNA-C, the restriction enzymes BamHI and SpeI were used to separate the DNA fragments of MDV and FBNYV DNA-C, and a pCAMBIA1303 vector treated with the same enzymes was obtained. The DNA fragments of MDV and FBNYV DNA-C were then ligated to the digested vector using T4 ligase. (ii) For DNA-M, the restriction enzymes KpnI and SpeI were used to separate the DNA fragments of MDV and FBNYV DNA-M, and a pCMBIA1303 vector was digested with the same enzymes. The DNA fragments were then ligated to the vector in the same manner. (iii) For DNA-N, the restriction enzymes BamHI and SpeI were used to separate the DNA fragments of MDV and FBNYV DNA-N, and a pCMBIA 1303 vector treated with the same enzymes was obtained. The DNA fragments were then ligated to the vector in the same manner. (iv) For DNA-U1, the restriction enzymes BamHI and SpeI were used to separate the DNA fragments of MDV and FBNYV DNA-U1, and a pCMBIA1303 vector treated with the same enzymes was obtained. The DNA fragments were then ligated to the vector in the same manner.

The resulting MDV recombinant plasmids (MDV-DNA-C-pCAMBIA1303, MDV-DNA-M-pCAMBIA1303, MDV-DNA-N-pCAMBIA1303, and MDV-DNA-U1-pCAMBIA1303) and FBNYV recombinant plasmids (FBNYV-DNA-C-pCAMBIA1303, FBNYV-DNA-M-pCAMBIA1303, FBNYV-DNA-N-pCAMBIA1303, and FBNYV-DNA-U1-pCAMBIA1303) were transformed back into the Escherichia coli DH5α strain, amplified, and extracted. Through restriction enzyme treatment, it was determined whether the recombinant plasmids were constructed correctly. Finally, the confirmed recombinant plasmids were transformed into Agrobacterium tumefaciens strain GV3101 to complete the infectious clones of MDV and FBNYV.

### Example 3. Verification of Infectivity of MDV and FBNYV DNA-C, DNA-M, DNA-N, and DNA-U1 Infectious Clones

### Example 3.1. Infectious Clones of MDV DNA-C, DNA-M, DNA-N, and DNA-U1

First, the MDV infectious clones (MDV DNA-C infectious clone, MDV DNA-M infectious clone, MDV DNA-N infectious clone, and MDV DNA-U1 infectious clone) produced in Example 2 was tested for infectivity, using Nicotiana benthamiana and cow pea.

N. benthamiana and cow pea were germinated and grown for two weeks. The apical parts of the prepared N. benthamiana and cow pea were wounded with a pin, and then inoculated with Agrobacterium cultures containing MDV DNA-C, DNA-M, DNA-N, and DNA-U1 infectious clones. The plants were cultivated for three more weeks to assess infectivity. After three weeks, genomic DNA was extracted from the young leaves of the plants, and PCR was performed using detection primer sets for each virus to confirm proper infection within the plant.

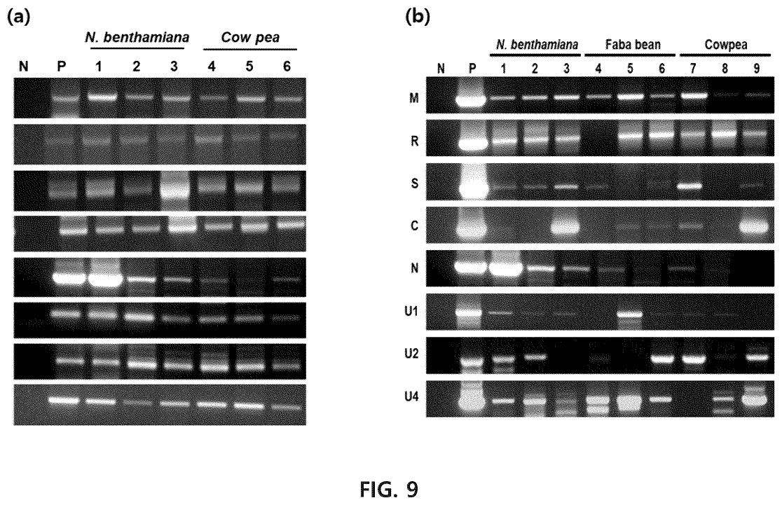

The results are presented in FIG. 9a.

Plants infected with MDV were observed to undergo symptoms such as leaf necrosis, chlorosis, and stunted growth, unlike healthy plants. The detection of MDV DNA-C, MDV DNA-M, MDV DNA-N, and MDV DNA-U1 within the symptomatic plants confirmed the successful infection by MDV (as shown in FIG. 9a).

### Example 3.2. Infectious Clones of FBNYV DNA-C, DNA-M, DNA-N, and DNA-U1

Next, the FBNYV infectious clones (FBNYV DNA-C infectious clone, FBNYV DNA-M infectious clone, FBNYV DNA-N infectious clone, and FBNYV DNA-U1 infectious clone) produced in Example 2 was tested for infectivity, using faba beans (Vicia faba), known as a host plant of FBNYV.

Initially, faba beans were germinated and grown for two weeks. The apical parts of the prepared faba beans were wounded with a pin, and then inoculated with Agrobacterium cultures containing FBNYV DNA-C, DNA-M, DNA-N, and DNA-U1 infectious clones. The plants were cultivated for three more weeks to assess infectivity. After three weeks, genomic DNA was extracted from the young leaves of the plants, and PCR was performed using detection primer sets for each virus to confirm proper infection within the plant.

The results are presented in FIG. 9b.

In plants infected with FBNYV, symptoms such as leaf necrosis, chlorosis, and stunted growth were observed unlike healthy plants. The detection of FBNYV DNA-C, DNA-M, DNA-N, and DNA-U1 within the symptomatic plants confirmed the successful infection by FBNYV (as shown in FIG. 9b).

What is claimed is:

1. A recombinant vector, comprising at least one selected from the group consisting of:
a nucleotide sequence of Nanovirus DNA-C represented by SEQ ID NO: 1;
a nucleotide sequence of Nanovirus DNA-M represented by SEQ ID NO: 2;
a nucleotide sequence of Nanovirus DNA-N represented by SEQ ID NO: 3; and
a nucleotide sequence of Nanovirus DNA-U1 represented by SEQ ID NO: 4.

2. A recombinant vector, comprising at least one selected from the group consisting of:
a nucleotide sequence of Nanovirus DNA-C represented by SEQ ID NO: 5;
a nucleotide sequence of Nanovirus DNA-M represented by SEQ ID NO: 6;
a nucleotide sequence of Nanovirus DNA-N represented by SEQ ID NO: 7; and
a nucleotide sequence of Nanovirus DNA-U1 represented by SEQ ID NO: 8.

3. A Nanovirus -infected plant comprising the recombinant vector of claim 1.

4. A Nanovirus -infected plant comprising the recombinant vector of claim 2.

5. The Nanovirus -infected plant of claim 3, further comprising a recombinant vector comprising a nucleotide sequence of Nanovirus DNA-R represented by SEQ ID NO: 9, a nucleotide sequence of Nanovirus DNA-S represented by SEQ ID NO: 10, or a combination thereof.

6. The Nanovirus -infected plant of claim 4, further comprising a recombinant vector comprising a nucleotide sequence of Nanovirus DNA-R represented by SEQ ID NO: 11, a nucleotide sequence of Nanovirus DNA-S represented by SEQ ID NO: 12, or a combination thereof.

7. A composition for diagnosis of Nanovirus infection, comprising:
(a) an agent for detecting Nanovirus DNA-C including the nucleotide sequence of SEQ ID NO: 1 or a fragment thereof, Nanovirus DNA-M including the nucleotide sequence of SEQ ID NO: 2 or a fragment thereof, Nanovirus DNA-N including the nucleotide sequence of SEQ ID NO: 3 or a fragment thereof, Nanovirus DNA-U1 including the nucleotide sequence of SEQ ID NO: 4 or a fragment thereof, or a combination thereof;
(b) an agent for detecting Nanovirus DNA-C including the nucleotide sequence of SEQ ID NO: 5 or a fragment thereof, Nanovirus DNA-M including the nucleotide sequence of SEQ ID NO: 6 or a fragment thereof, Nanovirus DNA-N including the nucleotide sequence of SEQ ID NO: 7 or a fragment thereof, Nanovirus DNA-U1 including the nucleotide sequence of SEQ ID NO: 8 or a fragment thereof, or a combination thereof; or
(c) a combination thereof.

8. The composition of claim 7, further comprising:
(a′) an agent for detecting DNA-R including the nucleotide sequence of SEQ ID NO: 9 or a fragment thereof, DNA-S including the nucleotide sequence of SEQ ID NO: 10 or a fragment thereof, or a combination thereof;
(b′) an agent for detecting DNA-R including the nucleotide sequence of SEQ ID NO: 11 or a fragment thereof, DNA-S including the nucleotide sequence of SEQ ID NO: 12 or a fragment thereof, or a combination thereof; or
(c′) a combination thereof.

9. The composition of claim 7, wherein the detecting agent comprises primers or a probe specifically binding to Nanovirus DNA-C, DNA-M, DNA-N, or DNA-U1.

10. The composition of claim 7, further comprising primers or a probe specifically binding to Nanovirus DNA-R or DNA-S.

11. A method for diagnosing Nanovirus infection in a plant, the method comprising the steps of:
contacting the diagnostic composition of claim 7 with a plant-derived sample and performing a nucleic acid amplification reaction; and
detecting amplified nucleic acid.

12. The method of claim 11, wherein the amplified nucleic acid is selected from:
(aa) Nanovirus DNA-C including the nucleotide sequence of SEQ ID NO: 1 or a fragment thereof, Nanovirus DNA-M including the nucleotide sequence of SEQ ID NO: 2 or a fragment thereof, Nanovirus DNA-N including the nucleotide sequence of SEQ ID NO: 3 or a fragment thereof, Nanovirus DNA-U1 including the nucleotide sequence of SEQ ID NO: 4 or a fragment thereof, or a combination thereof;
(bb) Nanovirus DNA-C including the nucleotide sequence of SEQ ID NO: 5 or a fragment thereof, Nanovirus DNA-M including the nucleotide sequence of SEQ ID NO: 6 or a fragment thereof, Nanovirus DNA-N including the nucleotide sequence of SEQ ID NO: 7 or a fragment thereof, Nanovirus DNA-U1 including the nucleotide sequence of SEQ ID NO: 8 or a fragment thereof, or a combination thereof; or
(cc) a combination thereof.

13. The method of claim 11, wherein the amplified nucleic acid further comprises:
(aa′) Nanovirus DNA-R including the nucleotide sequence of SEQ ID NO: 9 or a fragment thereof, Nanovirus DNA-S including the nucleotide sequence of SEQ ID NO: 10 or a fragment thereof, or a combination thereof;
(bb′) Nanovirus DNA-R including the nucleotide sequence of SEQ ID NO: 11 or a fragment thereof, Nanovirus DNA-S including the nucleotide sequence of SEQ ID NO: 12 or a fragment thereof, or a combination thereof; or
(cc′) a combination thereof.

14. A method for inducing a Nanovirus disease in a plant, comprising a step of transfecting the recombinant vector of claim 1 into the plant.

15. A method for inducing a Nanovirus disease in a plant, comprising a step of transfecting the recombinant vector of claim 2 into the plant.

16. The method of claim 14, further comprising a step of transfecting into the plant a recombinant vector comprising the nucleotide sequence of Nanovirus DNA-R, represented by SEQ ID NO: 9, a recombinant vector comprising the nucleotide sequence of Nanovirus DNA-S, represented by SEQ ID NO: 10, or a combination thereof.

17. The method of claim 15, further comprising a step of transfecting into the plant a recombinant vector comprising the nucleotide sequence of Nanovirus DNA-R, represented by SEQ ID NO: 11. a recombinant vector comprising the nucleotide sequence of Nanovirus DNA-S. represented by SEQ ID NO: 12. or a combination thereof.


SEQUENCE LISTING (36 sequences)

SEQ ID NO: 1 - DNA, 988 bp, synthetic construct
  source 1..988: mol_type = other DNA
ctggggcggggcttagtattacccccgccccaggctcagcggagtcatcacgtgagacgcacatgactattaaatagaagacataataccaagtggtatatggtccccactaatatatttgatttaaatgcacggaatatatgctttgcttcgtctcgaagcaaagtaaggaatcactgggaccgatattagctgtaacccatgtgccttcctttatttgtatggtggacattaatttgctttaagatagcgtacattggaagcattgttattgcttataaaacctgagccgctattgtcatttttgctgttccgtcgtgtttctattgtccattgatatttcatggcggatcctggatattatcaaggttatcaagacgacggcgatatcgatgcccagaagagacatcaagctctatatttgatcggcataattatactcatcatggtatgtattattattctttgggtttgtattatgttggcgtgttatattcctgggtttttgaagaagacaatggaaacgtggttaagttcatcatctatgatgaagaggcgtcttgctgcgactataacacgcacacccttcgaggcaacaggacctgagagagaactaaattgggatgctcgcagacaagcaaaccctgcaccaagtcagccttccaatggaggagtattttagagaatgattatagattgtaattatatattcatatcatcattctattgtaagtgtcgcaccatcaatgagaatttacagaaattcgctgtgtcgtaattgtgtttcgtatgagttgcagacttctgtatatgtttcaaggaagaagaaaactattcactatgccacgtgttcacatagaaatggtttatattattttttattactccgcgtagcggtatgtttgggcttttaagcccaattggggttaggcccaattcagtagccaatgaggaaggcttaatgacctcatatgatcgaggtgctgag

SEQ ID NO: 2 - DNA, 988 bp, synthetic construct; identical to SEQ ID NO: 1
  source 1..988: mol_type = other DNA

SEQ ID NO: 3 - DNA, 973 bp, synthetic construct
  source 1..973: mol_type = other DNA
acccccgccccaggatcagcggagtcatttagactcgctataagccgttagatgtgtagacacgtgtacaatcaggatctgtgattcgtgaagcgaatctgacggaagattgtacaatacgctaaactatatatgtacgcgtgtttaattgccattggtccatatagtagtggacatcacgcgttttcttttaacgcggtttactttataagtggggaccagtagttgctttgtttgcacgaaaggttaatcttttgtgttgaagaagcttcttcgttgctctataaatacaagcttcttcttcatcctcttcgtcatcattttctgcaactgcaaaaaatggcagattggttcgcgagtcctttgaagacatgtacacatgtctgcgattttccaacccttgctggtaatccatcacaagagattacttgctgtgatagtatgaagaataagttaaatgattcgaggaaggttttacttgtgagttgcagtgtgagcttcaatggtagtttctatggaggtaacagaaacgttcgtggtcagttgcagatttctatgttagaagacgacggtgtttgtagaccaattggttatgtcccaattggtggttacttgtaccataacgactatggatattacgaaggtgagaagacattcaaccttgatattgaatcccagtacttgaagaaggacgaagactacaataggaagtttattgtttcagttctcaacgagaatggtttagacagtttatgcgacttaaaggtttttattgttcatgctcttaggatcaaagtgtaataatcataattattgtaagattatgcaattgaataataatatcaggatgaataatttatttttaaatactctgcgtagctgtatgttaggcccaataggcccatttgttagtaagcccaaatgaatatgacgtcatctgatcccttgctgagctggggcggggcttagtatt

SEQ ID NO: 4 - DNA, 990 bp, synthetic construct
  source 1..990: mol_type = other DNA
ggggcggggcttagtattacccccgccccgggttcagagtcacgtacggagtgacttacagccgttggattttaaataagatggacgatcaggatctgtgattcgtgaagcgaatctgacggaagatcgtccgaagcttcgtggtagggcccccctattgctttatctttactttaataaagtaaagtaagatgctgtcccctactttattcgtttatgagttgtttacagctgtctttgcttcgtctcgaagcaaaggaccttctcatcgtctataaaagctgtgtttatgaagaagatgaattgttcatcttcttctttctcacaaaccatattgtttgtttcatcaaactaaagcttctattgaggttatgtctgcgaacgattcttcgttgtttgatgaggccaccgatgagttaatatcgtctgagaggaaattgatagccgtcgaatgccatgatgatgattctcaggtaattaatgttaaggttgaagatatcttttgcgatatgtccgacaaggtcgttattaaactgcagtttagattgtgttacaagtaccgaaagctattagatattactcttcttgggtgtcgtatgaaggttcatacagagttgaagacaccatctgaacgttctctgaagagtattttgcagaagaagatgaatgttatatgcgatgggaattatttgataggtattagaatgttttttataaatattaatcagttgattaatacttgtaagtggattaccagaactgaagatgtatatccaatatgtactttgtatcatgtgaatgatattcctgtaattgatagttaataatgaatatttgttttttgatcaaacacatgtattgataaatatattatttttaattactctgcgaagctatatgtctaggcccaataggcccaaatgctcttaagcccaatgaaattaacactgaagtgagtcagctgacgtcagctgactccttgatgacgta

SEQ ID NO: 5 - DNA, 993 bp, synthetic construct
  source 1..993: mol_type = other DNA
acccccgccccgggttcagagtcacgtacggagtgactaatggccattggattattatacacgtggacgatcaggatctgtgattcgtgaagcgaatctgacggaagatcgtccgaagcttcgtggtagggcccaagtgttgctttagtttgcttttttaaaaagtaaagctagatgcttttctctactttattcctttgaagtggaatacagctgtctttgcttcctcccgaagcaaaggattctttatctctctataaaaagctgtgttcttcttcgttgttcttacaacgaagatgggtctgaaatatttttctcatcttcctgaagagttgagagagaagattgttcgtgatcatctacaacaagagcgaaagaaagagtttcttgagaatgctatagaagatagttgcagaaggcatgtgtcgttattaaagtcagacccatcaccttcagagctgtattctctgagtaagtttttagattcattggctgattatgttggtaagcagtttaatacacgttgtttgattaaatggaagaaagacgttcctgctaatatcaagtttcaagttatggaagaacaacacctgcggttatatgggtttgttgatatggacgaccttttatgtcgtgagttattgccacctgaagaagatgatgatattacatacgaagatggtatgattgtcaactgcagtgagttggataaattatttgaggctctaggcattagagttgtttatattactgtcagtaagaattgtatttggactcctttgaacaaggatattgtaattaaatgaattgtgataataataaattattactaatgttatcatgaaagatatatttattcttttaaatactctgcgaagctatatgttaaggcccaataggcccaattgctcttaggcccaattaaattctctttgaagtgagtcagctgacatcagctgactccttgatgacgtaggggcggggcttagtatt

SEQ ID NO: 6 - DNA, 993 bp, synthetic construct; identical to SEQ ID NO: 5
  source 1..993: mol_type = other DNA

SEQ ID NO: 7 - DNA, 987 bp, synthetic construct
  source 1..987: mol_type = other DNA
ctggggcggggcttagtattacccccgccccaggttcagcggagtcatacggacgcaaggatagccattggattgttggacacgtgtacaatcaggatctgtgattcgtgaagcgaatctgacggaagattgtacactattatattattattattatacgcgtggcaagatattattggatgaagttaaaggtctacctttattcacgcgtattgcttttttaacgcgctttgcttttagagtgggccactatgttcctttatctttaagaaagcagatgctttcttgttcaacaagcttatgctttttatgtttataaataagcttctcgtcttcttcatggtattcatatctgaatacgaaaatggcagattggttttcgagtcctctcaaaacatgtacgcatgtttgtgattttccttctcttgctggtaatccacagcaagagattatgtgctgtgatagtatgaagaataagttgcaagattcgaggaaggtgttacttgttagttgcagtgtgagctttaatggtagtttctacggtggtaacagaaacgttcgaggtcaactacagatctcgatggttgaagacgatggtgtttgtaggcctataggttatgttcctattggtggatatctgtatcataacgattatggatattacgaaggtgcaaggacattcaacctggatctcgagtctcagtacttgaagaaggatgaagattacaacaggaagtttttagtaaccgttcttaatgagaatggtttagatagtcagtgtgatttgaaagtgtttattgttcattctattagaatcaaagtgtgattattgtatattgtattattatgtgaaggattaaatgttaatgatatatcaatacatattactaatgttttaataactctgcgaagctatatgttaaggcccaataggcccaatagctgttaggcccaattaattgtgacgtcacttgatcccttgctgag

SEQ ID NO: 8 - DNA, 987 bp, synthetic construct
  source 1..987: mol_type = other DNA
acccccgccccgggttcagagtcacgtacggagtgaataatagccattggattataagacacgtggacgatcaggatctgtgattcgtgaagcgaatctgacggaagatcgtccgcagcttcgtggtaggtcccaagtgttgctttagtttgcttttttaaaagtaaagttagatgcttttctctactttattcctttgaagtggaatacagctgtctttgcttcctctcgaagcaaaggatgctttatctctctataaaaagctgtgttcgtctgagaagacgaattgttcatttcatcttcttctacgaatcgtcgtctctatatcagcgacgttagtcttaatgggtgtttcaccagttcgtgattcatggcttgttgatgaggcgtccgatgagttaatagcatctgaaaggaagttgattgctgttgaatgtcatgatgatgattctcaggtaattaatgtcaaagttgaagatattgcccaggatataaatgacaagattgttttgaagttgcaatttagattatgttataagtacaggaagttgttagatattactcttctcggttgtcgtatgaaagtttatacccagttgaagaacacaatcttgaattcgttgaagagtattctccagaagaggatgaataatatatgtaatggtaattattgtataggcattagaatgttttttgttaatataaatcagattattaagagttgtaagtggattgttagtactgaagatgtgtatcctatttgtactttgtatcatatgagagatacggatgtatcttttgtaattaaatgaatgttatattaataaatttattatgaaagatatatttattcttttaaatactctgcgaagctatatgttaaggcccaataggcccaatagctctaaggcccaattaaattctctttgaagtgagtcagctgacatcagctgactccttgatgacgtaggggcggggcttagtatt

SEQ ID NO: 9 - DNA, 1002 bp, synthetic construct
  source 1..1002: mol_type = other DNA
ctggggcggggcttagtattacccccgccccaggatcagcggagtcatcacgtgacccgcacatgcctttagtctatatatagcccaagttgtaatattttatcattcatcaataaaatatggctcggcaagttatatgctggtgctttacattaaataatcctctctctcctctagttcttcatgaatcaatgaagtaccttgtttatcaaagagaacaaggtgaagctggaaatattcatttccagggttatattgagatgaagaaacgcacgtctcttgcaggtatgaagcgattagttccaggagcccattttgagaaaatgagggtaaaccaagaccaggctaaggcttacgctatgaaggaagacacccgtcttgaaggtccatgggagtatggggagttcatccccaccattgaagataagctcagggaagttatgaacgacatgaagattacaggtaagagacctattgagtatatagaagagtgctgtaatacatacgataaatctgcaagtactctaagggagttcagaggtgagttgaagaagaagaaggcaattttaagttgggagttgcagaggaagccatggatggacgaggtcgataccttgcttcaggagagagatggaagaagaatcatttgggtgtatggcccacaaggtggagaagggaaaacctcttacgctaagcatcttgtgaaaacgcgtgatgctttttattcgacaggtggaaagacagccgacattgcttttgcgtgggaccaccaagagttagtgcttttcgactttcctcgaagcttcgaggagtatgttaattatggagtaatagagcaattaaagaatgggattatccagtctggcaagtaccaaagtgtaattaagtatagtgattatgtggaggtgattgtatttgctaattttactccgcgtagcggtatgtttagtgatgataggattgtatatgtatatgcatgacgtcatatgatcccgtgctgag

SEQ ID NO: 10 - DNA, 994 bp, synthetic construct
  source 1..994: mol_type = other DNA
ctggggcggggcttagtattacccccgcaccaggatcagcggagtcatttagactcattataagccgttagatgtgtagacacgtggacgatcaggatctgtgattcgtgaagcgaatctgacggaagatcgtccgaaacttcgtggtagggccctatgttgctttatctttactttattaaagtaaagaatgatgctgtcccctactttataccttttgagttgattacagctgtctttgcttcgtctccaagcaaagcatattttctctctctataaaagctgagaaatgtattcgttgtactcacaacgaaaatggtaatgaattggaattggaatgggatgaagagacgacgaacacctcgtcgtggttatgccaggccgttcaagcctgttgttcctatcaccagggtcgttgttcatcaatctgctttgttgaagaaagatgaagtcgttggatgtgagatcaaaccagatggtgatgtttctcgttataagatgaggaaggttatgttaacctgtactttgaggatgcctccgggagaattagtcaattacatcatcgtcaagagcagttctcccattgttaattggtctgcagcttttaccgctcctgctctgttagtgaaagaaagttgccaagacatgatatcaatcattgctaaaggcaaggttgagtccaatggagttgcaggtactgattgcacaaagtccttcaataaatttattagattgggagccggtatcagccaaacccaacatttgtatgttgttatgtacactagtgttgctttgaaggctgtgttagaacatagagtttatattgaattgtaatgtaatgaagaacactatgaaataatgaaaccaagtatcatttaatcttaaaactccgcgtagcggtatgtttccgtgtttttgtttccaataatgcccttcattaatgaaggagaattgtcaaatatgaccttgtgacgtcacttgatcccgtgctgaa

SEQ ID NO: 11 - DNA, 1157 bp, synthetic construct
  source 1..1157: mol_type = other DNA
ggtacccgtcatgtgatcccttgctgagctggggcggggcttagtattacccccgccccaggttcagcggagtcatcacgtggattgcacgtgctattagtctatatatagcccaagttgtaatattttatcattcattgaataaaatatggctcggcaagttatatgctggtgctttacattaaataatcctctctctcctctttctcttcatgattcaatgaagtaccttgtttatcaaactgaacaaggtgaagctggaaatattcatttccagggttatattgaaatgaagaagcgtacatctcttgcaggtatgaagaagttaattcctggtgctcatttcgaaaagaggagaggtactcaaggagaagccagagcttatgcaatgaaggaagatacaaggattgaaggtccatgggagtatggagagttcattccaactattgaagataagctcagagaagttatgaatgatatgaagactacaggtaagagacctattgagtatatagaagagtgttgtaatacatatgataagtctgcaagtactttaagagagttcagaggtgaattgaagaagaagaaggctatttccagttgggagttgcagaggaagccatggatggacgaggtcgatgctttgcttcaggagagagatggaagacgaatcatttgggtgtatggcccacaaggtggagaagggaaaacctcttacgctaagcatcttgtaaagacgcgtgatgctttttattcgacaggtggaaagacagccgacattgcttttgcttgggaccaccaagagttagtgctcttcgactttccacgaagcttcgaggaatatgttaattacggagtaatagaacaattaaagaatggtattattcagtctggaaagtatcaaagtgtaattaaatatagtgattatgttgaagtgattgtatttgctaattttactccgcgtagcggtatgtttagtgaggataggattgtctatgtatatgcgtgacgtcatgtgatcccttgctgagctggggcggggcttagtattacccccgccccaggttcagcggagtcatcacgtggattgcacgtgctattagtctatatatagcccaagttgtaatattttatcattcattgaataaaatactagt

SEQ ID NO: 12 - DNA, 102204 bp, synthetic construct
  source 1..102204: mol_type = other DNA
ggatcctctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaatggcgagtacatggaattggtctggtaagaaaggaagaagaacactacgccgtccttatggtcgaccatacaagtcgtatgttccaacgacgcgagttgttgttcatcaatcagctgttttgaagaaagatgaggttgctggtacagaaatcaagcctgaaggtgatgtagttcgttataaaatgaagaaggtgatgctaacatgtactctgaggatggctcctggagagttagtgaattacgttattgttaaatgtaattcgcctattgttacttggtctgctgcgtttaccagtcctgctctcttggtgaaggagagttgtcaggatatgatcacaattattgcgaaaggcaaggtcgaatcgaatggtgttgctggtaccgattgtaccaagtcattcaatcgatttattcagttaggttctggaattactcaaactcagcatctatatgtggtgttatacaccagtgttgctctcaaagctgtgctagaacatacagtttatgtcgaagtgtaatctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaactagtggatcctctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaatggcgagtacatggaattggtctggtaagaaaggaagaagaacactacgccgtccttatggtcgaccatacaagtcgtatgttccaacgacgcgagttgttgttcatcaatcagctgttttgaagaaagatgaggttgctggtacagaaatcaagcctgaaggtgatgtagttcgttataaaatgaagaaggtgatgctaacatgtactctgaggatggctcctggagagttagtgaattacgttattgttaaatgtaattcgcctattgttacttggtctgctgcgtttaccagtcctgctctcttggtgaaggagagttgtcaggatatgatcacaattattgcgaaaggcaaggtcgaatcgaatggtgttgctggtaccgattgtaccaagtcattcaatcgatttattcagttaggttctggaattactcaaactcagcatctatatgtggtgttatacaccagtgttgctctcaaagctgtgctagaacatacagtttatgtcgaagtgtaatctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaactagtggatcctctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaatggcgagtacatggaattggtctggtaagaaaggaagaagaacactacgccgtccttatggtcgaccatacaagtcgtatgttccaacgacgcgagttgttgttcatcaatcagctgttttgaagaaagatgaggttgctggtacagaaatcaagcctgaaggtgatgtagttcgttataaaatgaagaaggtgatgctaacatgtactctgaggatggctcctggagagttagtgaattacgttattgttaaatgtaattcgcctattgttacttggtctgctgcgtttaccagtcctgctctcttggtgaaggagagttgtcaggatatgatcacaattattgcgaaaggcaaggtcgaatcgaatggtgttgctggtaccgattgtaccaagtcattcaatcgatttattcagttaggttctggaattactcaaactcagcatctatatgtggtgttatacaccagtgttgctctcaaagctgtgctagaacatacagtttatgtcgaagtgtaatctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaactagtggatcctctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaatggcgagtacatggaattggtctggtaagaaaggaagaagaacactacgccgtccttatggtcgaccatacaagtcgtatgttccaacgacgcgagttgttgttcatcaatcagctgttttgaagaaagatgaggttgctggtacagaaatcaagcctgaaggtgatgtagttcgttataaaatgaagaaggtgatgctaacatgtactctgaggatggctcctggagagttagtgaattacgttattgttaaatgtaattcgcctattgttacttggtctgctgcgtttaccagtcctgctctcttggtgaaggagagttgtcaggatatgatcacaattattgcgaaaggcaaggtcgaatcgaatggtgttgctggtaccgattgtaccaagtcattcaatcgatttattcagttaggttctggaattactcaaactcagcatctatatgtggtgttatacaccagtgttgctctcaaagctgtgctagaacatacagtttatgtcgaagtgtaatctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaactagtggatcctctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaatggcgagtacatggaattggtctggtaagaaaggaagaagaacactacgccgtccttatggtcgaccatacaagtcgtatgttccaacgacgcgagttgttgttcatcaatcagctgttttgaagaaagatgaggttgctggtacagaaatcaagcctgaaggtgatgtagttcgttataaaatgaagaaggtgatgctaacatgtactctgaggatggctcctggagagttagtgaattacgttattgttaaatgtaattcgcctattgttacttggtctgctgcgtttaccagtcctgctctcttggtgaaggagagttgtcaggatatgatcacaattattgcgaaaggcaaggtcgaatcgaatggtgttgctggtaccgattgtaccaagtcattcaatcgatttattcagttaggttctggaattactcaaactcagcatctatatgtggtgttatacaccagtgttgctctcaaagctgtgctagaacatacagtttatgtcgaagtgtaatctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaactagtggatcctctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaatggcgagtacatggaattggtctggtaagaaaggaagaagaacactacgccgtccttatggtcgaccatacaagtcgtatgttccaacgacgcgagttgttgttcatcaatcagctgttttgaagaaagatgaggttgctggtacagaaatcaagcctgaaggtgatgtagttcgttataaaatgaagaaggtgatgctaacatgtactctgaggatggctcctggagagttagtgaattacgttattgttaaatgtaattcgcctattgttacttggtctgctgcgtttaccagtcctgctctcttggtgaaggagagttgtcaggatatgatcacaattattgcgaaaggcaaggtcgaatcgaatggtgttgctggtaccgattgtaccaagtcattcaatcgatttattcagttaggttctggaattactcaaactcagcatctatatgtggtgttatacaccagtgttgctctcaaagctgtgctagaacatacagtttatgtcgaagtgtaatctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaactagtggatcctctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaatggcgagtacatggaattggtctggtaagaaaggaagaagaacactacgccgtccttatggtcgaccatacaagtcgtatgttccaacgacgcgagttgttgttcatcaatcagctgttttgaagaaagatgaggttgctggtacagaaatcaagcctgaaggtgatgtagttcgttataaaatgaagaaggtgatgctaacatgtactctgaggatggctcctggagagttagtgaattacgttattgttaaatgtaattcgcctattgttacttggtctgctgcgtttaccagtcctgctctcttggtgaaggagagttgtcaggatatgatcacaattattgcgaaaggcaaggtcgaatcgaatggtgttgctggtaccgattgtaccaagtcattcaatcgatttattcagttaggttctggaattactcaaactcagcatctatatgtggtgttatacaccagtgttgctctcaaagctgtgctagaacatacagtttatgtcgaagtgtaatctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaactagtggatcctctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaatggcgagtacatggaattggtctggtaagaaaggaagaagaacactacgccgtccttatggtcgaccatacaagtcgtatgttccaacgacgcgagttgttgttcatcaatcagctgttttgaagaaagatgaggttgctggtacagaaatcaagcctgaaggtgatgtagttcgttataaaatgaagaaggtgatgctaacatgtactctgaggatggctcctggagagttagtgaattacgttattgttaaatgtaattcgcctattgttacttggtctgctgcgtttaccagtcctgctctcttggtgaaggagagttgtcaggatatgatcacaattattgcgaaaggcaaggtcgaatcgaatggtgttgctggtaccgattgtaccaagtcattcaatcgatttattcagttaggttctggaattactcaaactcagcatctatatgtggtgttatacaccagtgttgctctcaaagctgtgctagaacatacagtttatgtcgaagtgtaatctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaactagtggatcctctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaatggcgagtacatggaattggtctggtaagaaaggaagaagaacactacgccgtccttatggtcgaccatacaagtcgtatgttccaacgacgcgagttgttgttcatcaatcagctgttttgaagaaagatgaggttgctggtacagaaatcaagcctgaaggtgatgtagttcgttataaaatgaagaaggtgatgctaacatgtactctgaggatggctcctggagagttagtgaattacgttattgttaaatgtaattcgcctattgttacttggtctgctgcgtttaccagtcctgctctcttggtgaaggagagttgtcaggatatgatcacaattattgcgaaaggcaaggtcgaatcgaatggtgttgctggtaccgattgtaccaagtcattcaatcgatttattcagttaggttctggaattactcaaactcagcatctatatgtggtgttatacaccagtgttgctctcaaagctgtgctagaacatacagtttatgtcgaagtgtaatctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaactagtggatcctctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaatggcgagtacatggaattggtctggtaagaaaggaagaagaacactacgccgtccttatggtcgaccatacaagtcgtatgttccaacgacgcgagttgttgttcatcaatcagctgttttgaagaaagatgaggttgctggtacagaaatcaagcctgaaggtgatgtagttcgttataaaatgaagaaggtgatgctaacatgtactctgaggatggctcctggagagttagtgaattacgttattgttaaatgtaattcgcctattgttacttggtctgctgcgtttaccagtcctgctctcttggtgaaggagagttgtcaggatatgatcacaattattgcgaaaggcaaggtcgaatcgaatggtgttgctggtaccgattgtaccaagtcattcaatcgatttattcagttaggttctggaattactcaaactcagcatctatatgtggtgttatacaccagtgttgctctcaaagctgtgctagaacatacagtttatgtcgaagtgtaatctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaactagtggatcctctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaatggcgagtacatggaattggtctggtaagaaaggaagaagaacactacgccgtccttatggtcgaccatacaagtcgtatgttccaacgacgcgagttgttgttcatcaatcagctgttttgaagaaagatgaggttgctggtacagaaatcaagcctgaaggtgatgtagttcgttataaaatgaagaaggtgatgctaacatgtactctgaggatggctcctggagagttagtgaattacgttattgttaaatgtaattcgcctattgttacttggtctgctgcgtttaccagtcctgctctcttggtgaaggagagttgtcaggatatgatcacaattattgcgaaaggcaaggtcgaatcgaatggtgttgctggtaccgattgtaccaagtcattcaatcgatttattcagttaggttctggaattactcaaactcagcatctatatgtggtgttatacaccagtgttgctctcaaagctgtgctagaacatacagtttatgtcgaagtgtaatctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaactagtggatcctctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaatggcgagtacatggaattggtctggtaagaaaggaagaagaacactacgccgtccttatggtcgaccatacaagtcgtatgttccaacgacgcgagttgttgttcatcaatcagctgttttgaagaaagatgaggttgctggtacagaaatcaagcctgaaggtgatgtagttcgttataaaatgaagaaggtgatgctaacatgtactctgaggatggctcctggagagttagtgaattacgttattgttaaatgtaattcgcctattgttacttggtctgctgcgtttaccagtcctgctctcttggtgaaggagagttgtcaggatatgatcacaattattgcgaaaggcaaggtcgaatcgaatggtgttgctggtaccgattgtaccaagtcattcaatcgatttattcagttaggttctggaattactcaaactcagcatctatatgtggtgttatacaccagtgttgctctcaaagctgtgctagaacatacagtttatgtcgaagtgtaatctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaactagtggatcctctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaatggcgagtacatggaattggtctggtaagaaaggaagaagaacactacgccgtccttatggtcgaccatacaagtcgtatgttccaacgacgcgagttgttgttcatcaatcagctgttttgaagaaagatgaggttgctggtacagaaatcaagcctgaaggtgatgtagttcgttataaaatgaagaaggtgatgctaacatgtactctgaggatggctcctggagagttagtgaattacgttattgttaaatgtaattcgcctattgttacttggtctgctgcgtttaccagtcctgctctcttggtgaaggagagttgtcaggatatgatcacaattattgcgaaaggcaaggtcgaatcgaatggtgttgctggtaccgattgtaccaagtcattcaatcgatttattcagttaggttctggaattactcaaactcagcatctatatgtggtgttatacaccagtgttgctctcaaagctgtgctagaacatacagtttatgtcgaagtgtaatctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaactagtggatcctctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaatggcgagtacatggaattggtctggtaagaaaggaagaagaacactacgccgtccttatggtcgaccatacaagtcgtatgttccaacgacgcgagttgttgttcatcaatcagctgttttgaagaaagatgaggttgctggtacagaaatcaagcctgaaggtgatgtagttcgttataaaatgaagaaggtgatgctaacatgtactctgaggatggctcctggagagttagtgaattacgttattgttaaatgtaattcgcctattgttacttggtctgctgcgtttaccagtcctgctctcttggtgaaggagagttgtcaggatatgatcacaattattgcgaaaggcaaggtcgaatcgaatggtgttgctggtaccgattgtaccaagtcattcaatcgatttattcagttaggttctggaattactcaaactcagcatctatatgtggtgttatacaccagtgttgctctcaaagctgtgctagaacatacagtttatgtcgaagtgtaatctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaactagtggatcctctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaatggcgagtacatggaattggtctggtaagaaaggaagaagaacactacgccgtccttatggtcgaccatacaagtcgtatgttccaacgacgcgagttgttgttcatcaatcagctgttttgaagaaagatgaggttgctggtacagaaatcaagcctgaaggtgatgtagttcgttataaaatgaagaaggtgatgctaacatgtactctgaggatggctcctggagagttagtgaattacgttattgttaaatgtaattcgcctattgttacttggtctgctgcgtttaccagtcctgctctcttggtgaaggagagttgtcaggatatgatcacaattattgcgaaaggcaaggtcgaatcgaatggtgttgctggtaccgattgtaccaagtcattcaatcgatttattcagttaggttctggaattactcaaactcagcatctatatgtggtgttatacaccagtgttgctctcaaagctgtgctagaacatacagtttatgtcgaagtgtaatctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaactagtggatcctctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaatggcgagtacatggaattggtctggtaagaaaggaagaagaacactacgccgtccttatggtcgaccatacaagtcgtatgttccaacgacgcgagttgttgttcatcaatcagctgttttgaagaaagatgaggttgctggtacagaaatcaagcctgaaggtgatgtagttcgttataaaatgaagaaggtgatgctaacatgtactctgaggatggctcctggagagttagtgaattacgttattgttaaatgtaattcgcctattgttacttggtctgctgcgtttaccagtcctgctctcttggtgaaggagagttgtcaggatatgatcacaattattgcgaaaggcaaggtcgaatcgaatggtgttgctggtaccgattgtaccaagtcattcaatcgatttattcagttaggttctggaattactcaaactcagcatctatatgtggtgttatacaccagtgttgctctcaaagctgtgctagaacatacagtttatgtcgaagtgtaatctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaactagtggatcctctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaatggcgagtacatggaattggtctggtaagaaaggaagaagaacactacgccgtccttatggtcgaccatacaagtcgtatgttccaacgacgcgagttgttgttcatcaatcagctgttttgaagaaagatgaggttgctggtacagaaatcaagcctgaaggtgatgtagttcgttataaaatgaagaaggtgatgctaacatgtactctgaggatggctcctggagagttagtgaattacgttattgttaaatgtaattcgcctattgttacttggtctgctgcgtttaccagtcctgctctcttggtgaaggagagttgtcaggatatgatcacaattattgcgaaaggcaaggtcgaatcgaatggtgttgctggtaccgattgtaccaagtcattcaatcgatttattcagttaggttctggaattactcaaactcagcatctatatgtggtgttatacaccagtgttgctctcaaagctgtgctagaacatacagtttatgtcgaagtgtaatctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaactagtggatcctctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaatggcgagtacatggaattggtctggtaagaaaggaagaagaacactacgccgtccttatggtcgaccatacaagtcgtatgttccaacgacgcgagttgttgttcatcaatcagctgttttgaagaaagatgaggttgctggtacagaaatcaagcctgaaggtgatgtagttcgttataaaatgaagaaggtgatgctaacatgtactctgaggatggctcctggagagttagtgaattacgttattgttaaatgtaattcgcctattgttacttggtctgctgcgtttaccagtcctgctctcttggtgaaggagagttgtcaggatatgatcacaattattgcgaaaggcaaggtcgaatcgaatggtgttgctggtaccgattgtaccaagtcattcaatcgatttattcagttaggttctggaattactcaaactcagcatctatatgtggtgttatacaccagtgttgctctcaaagctgtgctagaacatacagtttatgtcgaagtgtaatctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaactagtggatcctctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaatggcgagtacatggaattggtctggtaagaaaggaagaagaacactacgccgtccttatggtcgaccatacaagtcgtatgttccaacgacgcgagttgttgttcatcaatcagctgttttgaagaaagatgaggttgctggtacagaaatcaagcctgaaggtgatgtagttcgttataaaatgaagaaggtgatgctaacatgtactctgaggatggctcctggagagttagtgaattacgttattgttaaatgtaattcgcctattgttacttggtctgctgcgtttaccagtcctgctctcttggtgaaggagagttgtcaggatatgatcacaattattgcgaaaggcaaggtcgaatcgaatggtgttgctggtaccgattgtaccaagtcattcaatcgatttattcagttaggttctggaattactcaaactcagcatctatatgtggtgttatacaccagtgttgctctcaaagctgtgctagaacatacagtttatgtcgaagtgtaatctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaactagtggatcctctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaatggcgagtacatggaattggtctggtaagaaaggaagaagaacactacgccgtccttatggtcgaccatacaagtcgtatgttccaacgacgcgagttgttgttcatcaatcagctgttttgaagaaagatgaggttgctggtacagaaatcaagcctgaaggtgatgtagttcgttataaaatgaagaaggtgatgctaacatgtactctgaggatggctcctggagagttagtgaattacgttattgttaaatgtaattcgcctattgttacttggtctgctgcgtttaccagtcctgctctcttggtgaaggagagttgtcaggatatgatcacaattattgcgaaaggcaaggtcgaatcgaatggtgttgctggtaccgattgtaccaagtcattcaatcgatttattcagttaggttctggaattactcaaactcagcatctatatgtggtgttatacaccagtgttgctctcaaagctgtgctagaacatacagtttatgtcgaagtgtaatctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaactagtggatcctctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaatggcgagtacatggaattggtctggtaagaaaggaagaagaacactacgccgtccttatggtcgaccatacaagtcgtatgttccaacgacgcgagttgttgttcatcaatcagctgttttgaagaaagatgaggttgctggtacagaaatcaagcctgaaggtgatgtagttcgttataaaatgaagaaggtgatgctaacatgtactctgaggatggctcctggagagttagtgaattacgttattgttaaatgtaattcgcctattgttacttggtctgctgcgtttaccagtcctgctctcttggtgaaggagagttgtcaggatatgatcacaattattgcgaaaggcaaggtcgaatcgaatggtgttgctggtaccgattgtaccaagtcattcaatcgatttattcagttaggttctggaattactcaaactcagcatctatatgtggtgttatacaccagtgttgctctcaaagctgtgctagaacatacagtttatgtcgaagtgtaatctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaactagtggatcctctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaatggcgagtacatggaattggtctggtaagaaaggaagaagaacactacgccgtccttatggtcgaccatacaagtcgtatgttccaacgacgcgagttgttgttcatcaatcagctgttttgaagaaagatgaggttgctggtacagaaatcaagcctgaaggtgatgtagttcgttataaaatgaagaaggtgatgctaacatgtactctgaggatggctcctggagagttagtgaattacgttattgttaaatgtaattcgcctattgttacttggtctgctgcgtttaccagtcctgctctcttggtgaaggagagttgtcaggatatgatcacaattattgcgaaaggcaaggtcgaatcgaatggtgttgctggtaccgattgtaccaagtcattcaatcgatttattcagttaggttctggaattactcaaactcagcatctatatgtggtgttatacaccagtgttgctctcaaagctgtgctagaacatacagtttatgtcgaagtgtaatctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaactagtggatcctctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaatggcgagtacatggaattggtctggtaagaaaggaagaagaacactacgccgtccttatggtcgaccatacaagtcgtatgttccaacgacgcgagttgttgttcatcaatcagctgttttgaagaaagatgaggttgctggtacagaaatcaagcctgaaggtgatgtagttcgttataaaatgaagaaggtgatgctaacatgtactctgaggatggctcctggagagttagtgaattacgttattgttaaatgtaattcgcctattgttacttggtctgctgcgtttaccagtcctgctctcttggtgaaggagagttgtcaggatatgatcacaattattgcgaaaggcaaggtcgaatcgaatggtgttgctggtaccgattgtaccaagtcattcaatcgatttattcagttaggttctggaattactcaaactcagcatctatatgtggtgttatacaccagtgttgctctcaaagctgtgctagaacatacagtttatgtcgaagtgtaatctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaactagtggatcctctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaatggcgagtacatggaattggtctggtaagaaaggaagaagaacactacgccgtccttatggtcgaccatacaagtcgtatgttccaacgacgcgagttgttgttcatcaatcagctgttttgaagaaagatgaggttgctggtacagaaatcaagcctgaaggtgatgtagttcgttataaaatgaagaaggtgatgctaacatgtactctgaggatggctcctggagagttagtgaattacgttattgttaaatgtaattcgcctattgttacttggtctgctgcgtttaccagtcctgctctcttggtgaaggagagttgtcaggatatgatcacaattattgcgaaaggcaaggtcgaatcgaatggtgttgctggtaccgattgtaccaagtcattcaatcgatttattcagttaggttctggaattactcaaactcagcatctatatgtggtgttatacaccagtgttgctctcaaagctgtgctagaacatacagtttatgtcgaagtgtaatctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaactagtggatcctctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaatggcgagtacatggaattggtctggtaagaaaggaagaagaacactacgccgtccttatggtcgaccatacaagtcgtatgttccaacgacgcgagttgttgttcatcaatcagctgttttgaagaaagatgaggttgctggtacagaaatcaagcctgaaggtgatgtagttcgttataaaatgaagaaggtgatgctaacatgtactctgaggatggctcctggagagttagtgaattacgttattgttaaatgtaattcgcctattgttacttggtctgctgcgtttaccagtcctgctctcttggtgaaggagagttgtcaggatatgatcacaattattgcgaaaggcaaggtcgaatcgaatggtgttgctggtaccgattgtaccaagtcattcaatcgatttattcagttaggttctggaattactcaaactcagcatctatatgtggtgttatacaccagtgttgctctcaaagctgtgctagaacatacagtttatgtcgaagtgtaatctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaactagtggatcctctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaatggcgagtacatggaattggtctggtaagaaaggaagaagaacactacgccgtccttatggtcgaccatacaagtcgtatgttccaacgacgcgagttgttgttcatcaatcagctgttttgaagaaagatgaggttgctggtacagaaatcaagcctgaaggtgatgtagttcgttataaaatgaagaaggtgatgctaacatgtactctgaggatggctcctggagagttagtgaattacgttattgttaaatgtaattcgcctattgttacttggtctgctgcgtttaccagtcctgctctcttggtgaaggagagttgtcaggatatgatcacaattattgcgaaaggcaaggtcgaatcgaatggtgttgctggtaccgattgtaccaagtcattcaatcgatttattcagttaggttctggaattactcaaactcagcatctatatgtggtgttatacaccagtgttgctctcaaagctgtgctagaacatacagtttatgtcgaagtgtaatctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaactagtggatcctctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaatggcgagtacatggaattggtctggtaagaaaggaagaagaacactacgccgtccttatggtcgaccatacaagtcgtatgttccaacgacgcgagttgttgttcatcaatcagctgttttgaagaaagatgaggttgctggtacagaaatcaagcctgaaggtgatgtagttcgttataaaatgaagaaggtgatgctaacatgtactctgaggatggctcctggagagttagtgaattacgttattgttaaatgtaattcgcctattgttacttggtctgctgcgtttaccagtcctgctctcttggtgaaggagagttgtcaggatatgatcacaattattgcgaaaggcaaggtcgaatcgaatggtgttgctggtaccgattgtaccaagtcattcaatcgatttattcagttaggttctggaattactcaaactcagcatctatatgtggtgttatacaccagtgttgctctcaaagctgtgctagaacatacagtttatgtcgaagtgtaatctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaactagtggatcctctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaatggcgagtacatggaattggtctggtaagaaaggaagaagaacactacgccgtccttatggtcgaccatacaagtcgtatgttccaacgacgcgagttgttgttcatcaatcagctgttttgaagaaagatgaggttgctggtacagaaatcaagcctgaaggtgatgtagttcgttataaaatgaagaaggtgatgctaacatgtactctgaggatggctcctggagagttagtgaattacgttattgttaaatgtaattcgcctattgttacttggtctgctgcgtttaccagtcctgctctcttggtgaaggagagttgtcaggatatgatcacaattattgcgaaaggcaaggtcgaatcgaatggtgttgctggtaccgattgtaccaagtcattcaatcgatttattcagttaggttctggaattactcaaactcagcatctatatgtggtgttatacaccagtgttgctctcaaagctgtgctagaacatacagtttatgtcgaagtgtaatctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaactagtggatcctctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaatggcgagtacatggaattggtctggtaagaaaggaagaagaacactacgccgtccttatggtcgaccatacaagtcgtatgttccaacgacgcgagttgttgttcatcaatcagctgttttgaagaaagatgaggttgctggtacagaaatcaagcctgaaggtgatgtagttcgttataaaatgaagaaggtgatgctaacatgtactctgaggatggctcctggagagttagtgaattacgttattgttaaatgtaattcgcctattgttacttggtctgctgcgtttaccagtcctgctctcttggtgaaggagagttgtcaggatatgatcacaattattgcgaaaggcaaggtcgaatcgaatggtgttgctggtaccgattgtaccaagtcattcaatcgatttattcagttaggttctggaattactcaaactcagcatctatatgtggtgttatacaccagtgttgctctcaaagctgtgctagaacatacagtttatgtcgaagtgtaatctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaactagtggatcctctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaatggcgagtacatggaattggtctggtaagaaaggaagaagaacactacgccgtccttatggtcgaccatacaagtcgtatgttccaacgacgcgagttgttgttcatcaatcagctgttttgaagaaagatgaggttgctggtacagaaatcaagcctgaaggtgatgtagttcgttataaaatgaagaaggtgatgctaacatgtactctgaggatggctcctggagagttagtgaattacgttattgttaaatgtaattcgcctattgttacttggtctgctgcgtttaccagtcctgctctcttggtgaaggagagttgtcaggatatgatcacaattattgcgaaaggcaaggtcgaatcgaatggtgttgctggtaccgattgtaccaagtcattcaatcgatttattcagttaggttctggaattactcaaactcagcatctatatgtggtgttatacaccagtgttgctctcaaagctgtgctagaacatacagtttatgtcgaagtgtaatctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaactagtggatcctctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaatggcgagtacatggaattggtctggtaagaaaggaagaagaacactacgccgtccttatggtcgaccatacaagtcgtatgttccaacgacgcgagttgttgttcatcaatcagctgttttgaagaaagatgaggttgctggtacagaaatcaagcctgaaggtgatgtagttcgttataaaatgaagaaggtgatgctaacatgtactctgaggatggctcctggagagttagtgaattacgttattgttaaatgtaattcgcctattgttacttggtctgctgcgtttaccagtcctgctctcttggtgaaggagagttgtcaggatatgatcacaattattgcgaaaggcaaggtcgaatcgaatggtgttgctggtaccgattgtaccaagtcattcaatcgatttattcagttaggttctggaattactcaaactcagcatctatatgtggtgttatacaccagtgttgctctcaaagctgtgctagaacatacagtttatgtcgaagtgtaatctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaactagtggatcctctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaatggcgagtacatggaattggtctggtaagaaaggaagaagaacactacgccgtccttatggtcgaccatacaagtcgtatgttccaacgacgcgagttgttgttcatcaatcagctgttttgaagaaagatgaggttgctggtacagaaatcaagcctgaaggtgatgtagttcgttataaaatgaagaaggtgatgctaacatgtactctgaggatggctcctggagagttagtgaattacgttattgttaaatgtaattcgcctattgttacttggtctgctgcgtttaccagtcctgctctcttggtgaaggagagttgtcaggatatgatcacaattattgcgaaaggcaaggtcgaatcgaatggtgttgctggtaccgattgtaccaagtcattcaatcgatttattcagttaggttctggaattactcaaactcagcatctatatgtggtgttatacaccagtgttgctctcaaagctgtgctagaacatacagtttatgtcgaagtgtaatctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaactagtggatcctctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaatggcgagtacatggaattggtctggtaagaaaggaagaagaacactacgccgtccttatggtcgaccatacaagtcgtatgttccaacgacgcgagttgttgttcatcaatcagctgttttgaagaaagatgaggttgctggtacagaaatcaagcctgaaggtgatgtagttcgttataaaatgaagaaggtgatgctaacatgtactctgaggatggctcctggagagttagtgaattacgttattgttaaatgtaattcgcctattgttacttggtctgctgcgtttaccagtcctgctctcttggtgaaggagagttgtcaggatatgatcacaattattgcgaaaggcaaggtcgaatcgaatggtgttgctggtaccgattgtaccaagtcattcaatcgatttattcagttaggttctggaattactcaaactcagcatctatatgtggtgttatacaccagtgttgctctcaaagctgtgctagaacatacagtttatgtcgaagtgtaatctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaactagtggatcctctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaatggcgagtacatggaattggtctggtaagaaaggaagaagaacactacgccgtccttatggtcgaccatacaagtcgtatgttccaacgacgcgagttgttgttcatcaatcagctgttttgaagaaagatgaggttgctggtacagaaatcaagcctgaaggtgatgtagttcgttataaaatgaagaaggtgatgctaacatgtactctgaggatggctcctggagagttagtgaattacgttattgttaaatgtaattcgcctattgttacttggtctgctgcgtttaccagtcctgctctcttggtgaaggagagttgtcaggatatgatcacaattattgcgaaaggcaaggtcgaatcgaatggtgttgctggtaccgattgtaccaagtcattcaatcgatttattcagttaggttctggaattactcaaactcagcatctatatgtggtgttatacaccagtgttgctctcaaagctgtgctagaacatacagtttatgtcgaagtgtaatctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaactagtggatcctctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaatggcgagtacatggaattggtctggtaagaaaggaagaagaacactacgccgtccttatggtcgaccatacaagtcgtatgttccaacgacgcgagttgttgttcatcaatcagctgttttgaagaaagatgaggttgctggtacagaaatcaagcctgaaggtgatgtagttcgttataaaatgaagaaggtgatgctaacatgtactctgaggatggctcctggagagttagtgaattacgttattgttaaatgtaattcgcctattgttacttggtctgctgcgtttaccagtcctgctctcttggtgaaggagagttgtcaggatatgatcacaattattgcgaaaggcaaggtcgaatcgaatggtgttgctggtaccgattgtaccaagtcattcaatcgatttattcagttaggttctggaattactcaaactcagcatctatatgtggtgttatacaccagtgttgctctcaaagctgtgctagaacatacagtttatgtcgaagtgtaatctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaactagtggatcctctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaatggcgagtacatggaattggtctggtaagaaaggaagaagaacactacgccgtccttatggtcgaccatacaagtcgtatgttccaacgacgcgagttgttgttcatcaatcagctgttttgaagaaagatgaggttgctggtacagaaatcaagcctgaaggtgatgtagttcgttataaaatgaagaaggtgatgctaacatgtactctgaggatggctcctggagagttagtgaattacgttattgttaaatgtaattcgcctattgttacttggtctgctgcgtttaccagtcctgctctcttggtgaaggagagttgtcaggatatgatcacaattattgcgaaaggcaaggtcgaatcgaatggtgttgctggtaccgattgtaccaagtcattcaatcgatttattcagttaggttctggaattactcaaactcagcatctatatgtggtgttatacaccagtgttgctctcaaagctgtgctagaacatacagtttatgtcgaagtgtaatctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaactagtggatcctctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaatggcgagtacatggaattggtctggtaagaaaggaagaagaacactacgccgtccttatggtcgaccatacaagtcgtatgttccaacgacgcgagttgttgttcatcaatcagctgttttgaagaaagatgaggttgctggtacagaaatcaagcctgaaggtgatgtagttcgttataaaatgaagaaggtgatgctaacatgtactctgaggatggctcctggagagttagtgaattacgttattgttaaatgtaattcgcctattgttacttggtctgctgcgtttaccagtcctgctctcttggtgaaggagagttgtcaggatatgatcacaattattgcgaaaggcaaggtcgaatcgaatggtgttgctggtaccgattgtaccaagtcattcaatcgatttattcagttaggttctggaattactcaaactcagcatctatatgtggtgttatacaccagtgttgctctcaaagctgtgctagaacatacagtttatgtcgaagtgtaatctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaactagtggatcctctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaatggcgagtacatggaattggtctggtaagaaaggaagaagaacactacgccgtccttatggtcgaccatacaagtcgtatgttccaacgacgcgagttgttgttcatcaatcagctgttttgaagaaagatgaggttgctggtacagaaatcaagcctgaaggtgatgtagttcgttataaaatgaagaaggtgatgctaacatgtactctgaggatggctcctggagagttagtgaattacgttattgttaaatgtaattcgcctattgttacttggtctgctgcgtttaccagtcctgctctcttggtgaaggagagttgtcaggatatgatcacaattattgcgaaaggcaaggtcgaatcgaatggtgttgctggtaccgattgtaccaagtcattcaatcgatttattcagttaggttctggaattactcaaactcagcatctatatgtggtgttatacaccagtgttgctctcaaagctgtgctagaacatacagtttatgtcgaagtgtaatctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaactagtggatcctctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaatggcgagtacatggaattggtctggtaagaaaggaagaagaacactacgccgtccttatggtcgaccatacaagtcgtatgttccaacgacgcgagttgttgttcatcaatcagctgttttgaagaaagatgaggttgctggtacagaaatcaagcctgaaggtgatgtagttcgttataaaatgaagaaggtgatgctaacatgtactctgaggatggctcctggagagttagtgaattacgttattgttaaatgtaattcgcctattgttacttggtctgctgcgtttaccagtcctgctctcttggtgaaggagagttgtcaggatatgatcacaattattgcgaaaggcaaggtcgaatcgaatggtgttgctggtaccgattgtaccaagtcattcaatcgatttattcagttaggttctggaattactcaaactcagcatctatatgtggtgttatacaccagtgttgctctcaaagctgtgctagaacatacagtttatgtcgaagtgtaatctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaactagtggatcctctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaatggcgagtacatggaattggtctggtaagaaaggaagaagaacactacgccgtccttatggtcgaccatacaagtcgtatgttccaacgacgcgagttgttgttcatcaatcagctgttttgaagaaagatgaggttgctggtacagaaatcaagcctgaaggtgatgtagttcgttataaaatgaagaaggtgatgctaacatgtactctgaggatggctcctggagagttagtgaattacgttattgttaaatgtaattcgcctattgttacttggtctgctgcgtttaccagtcctgctctcttggtgaaggagagttgtcaggatatgatcacaattattgcgaaaggcaaggtcgaatcgaatggtgttgctggtaccgattgtaccaagtcattcaatcgatttattcagttaggttctggaattactcaaactcagcatctatatgtggtgttatacaccagtgttgctctcaaagctgtgctagaacatacagtttatgtcgaagtgtaatctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaactagtggatcctctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaatggcgagtacatggaattggtctggtaagaaaggaagaagaacactacgccgtccttatggtcgaccatacaagtcgtatgttccaacgacgcgagttgttgttcatcaatcagctgttttgaagaaagatgaggttgctggtacagaaatcaagcctgaaggtgatgtagttcgttataaaatgaagaaggtgatgctaacatgtactctgaggatggctcctggagagttagtgaattacgttattgttaaatgtaattcgcctattgttacttggtctgctgcgtttaccagtcctgctctcttggtgaaggagagttgtcaggatatgatcacaattattgcgaaaggcaaggtcgaatcgaatggtgttgctggtaccgattgtaccaagtcattcaatcgatttattcagttaggttctggaattactcaaactcagcatctatatgtggtgttatacaccagtgttgctctcaaagctgtgctagaacatacagtttatgtcgaagtgtaatctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaactagtggatcctctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaatggcgagtacatggaattggtctggtaagaaaggaagaagaacactacgccgtccttatggtcgaccatacaagtcgtatgttccaacgacgcgagttgttgttcatcaatcagctgttttgaagaaagatgaggttgctggtacagaaatcaagcctgaaggtgatgtagttcgttataaaatgaagaaggtgatgctaacatgtactctgaggatggctcctggagagttagtgaattacgttattgttaaatgtaattcgcctattgttacttggtctgctgcgtttaccagtcctgctctcttggtgaaggagagttgtcaggatatgatcacaattattgcgaaaggcaaggtcgaatcgaatggtgttgctggtaccgattgtaccaagtcattcaatcgatttattcagttaggttctggaattactcaaactcagcatctatatgtggtgttatacaccagtgttgctctcaaagctgtgctagaacatacagtttatgtcgaagtgtaatctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaactagtggatcctctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaatggcgagtacatggaattggtctggtaagaaaggaagaagaacactacgccgtccttatggtcgaccatacaagtcgtatgttccaacgacgcgagttgttgttcatcaatcagctgttttgaagaaagatgaggttgctggtacagaaatcaagcctgaaggtgatgtagttcgttataaaatgaagaaggtgatgctaacatgtactctgaggatggctcctggagagttagtgaattacgttattgttaaatgtaattcgcctattgttacttggtctgctgcgtttaccagtcctgctctcttggtgaaggagagttgtcaggatatgatcacaattattgcgaaaggcaaggtcgaatcgaatggtgttgctggtaccgattgtaccaagtcattcaatcgatttattcagttaggttctggaattactcaaactcagcatctatatgtggtgttatacaccagtgttgctctcaaagctgtgctagaacatacagtttatgtcgaagtgtaatctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaactagtggatcctctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaatggcgagtacatggaattggtctggtaagaaaggaagaagaacactacgccgtccttatggtcgaccatacaagtcgtatgttccaacgacgcgagttgttgttcatcaatcagctgttttgaagaaagatgaggttgctggtacagaaatcaagcctgaaggtgatgtagttcgttataaaatgaagaaggtgatgctaacatgtactctgaggatggctcctggagagttagtgaattacgttattgttaaatgtaattcgcctattgttacttggtctgctgcgtttaccagtcctgctctcttggtgaaggagagttgtcaggatatgatcacaattattgcgaaaggcaaggtcgaatcgaatggtgttgctggtaccgattgtaccaagtcattcaatcgatttattcagttaggttctggaattactcaaactcagcatctatatgtggtgttatacaccagtgttgctctcaaagctgtgctagaacatacagtttatgtcgaagtgtaatctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaactagtggatcctctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaatggcgagtacatggaattggtctggtaagaaaggaagaagaacactacgccgtccttatggtcgaccatacaagtcgtatgttccaacgacgcgagttgttgttcatcaatcagctgttttgaagaaagatgaggttgctggtacagaaatcaagcctgaaggtgatgtagttcgttataaaatgaagaaggtgatgctaacatgtactctgaggatggctcctggagagttagtgaattacgttattgttaaatgtaattcgcctattgttacttggtctgctgcgtttaccagtcctgctctcttggtgaaggagagttgtcaggatatgatcacaattattgcgaaaggcaaggtcgaatcgaatggtgttgctggtaccgattgtaccaagtcattcaatcgatttattcagttaggttctggaattactcaaactcagcatctatatgtggtgttatacaccagtgttgctctcaaagctgtgctagaacatacagtttatgtcgaagtgtaatctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaactagtggatcctctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaatggcgagtacatggaattggtctggtaagaaaggaagaagaacactacgccgtccttatggtcgaccatacaagtcgtatgttccaacgacgcgagttgttgttcatcaatcagctgttttgaagaaagatgaggttgctggtacagaaatcaagcctgaaggtgatgtagttcgttataaaatgaagaaggtgatgctaacatgtactctgaggatggctcctggagagttagtgaattacgttattgttaaatgtaattcgcctattgttacttggtctgctgcgtttaccagtcctgctctcttggtgaaggagagttgtcaggatatgatcacaattattgcgaaaggcaaggtcgaatcgaatggtgttgctggtaccgattgtaccaagtcattcaatcgatttattcagttaggttctggaattactcaaactcagcatctatatgtggtgttatacaccagtgttgctctcaaagctgtgctagaacatacagtttatgtcgaagtgtaatctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaactagtggatcctctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaatggcgagtacatggaattggtctggtaagaaaggaagaagaacactacgccgtccttatggtcgaccatacaagtcgtatgttccaacgacgcgagttgttgttcatcaatcagctgttttgaagaaagatgaggttgctggtacagaaatcaagcctgaaggtgatgtagttcgttataaaatgaagaaggtgatgctaacatgtactctgaggatggctcctggagagttagtgaattacgttattgttaaatgtaattcgcctattgttacttggtctgctgcgtttaccagtcctgctctcttggtgaaggagagttgtcaggatatgatcacaattattgcgaaaggcaaggtcgaatcgaatggtgttgctggtaccgattgtaccaagtcattcaatcgatttattcagttaggttctggaattactcaaactcagcatctatatgtggtgttatacaccagtgttgctctcaaagctgtgctagaacatacagtttatgtcgaagtgtaatctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaactagtggatcctctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaatggcgagtacatggaattggtctggtaagaaaggaagaagaacactacgccgtccttatggtcgaccatacaagtcgtatgttccaacgacgcgagttgttgttcatcaatcagctgttttgaagaaagatgaggttgctggtacagaaatcaagcctgaaggtgatgtagttcgttataaaatgaagaaggtgatgctaacatgtactctgaggatggctcctggagagttagtgaattacgttattgttaaatgtaattcgcctattgttacttggtctgctgcgtttaccagtcctgctctcttggtgaaggagagttgtcaggatatgatcacaattattgcgaaaggcaaggtcgaatcgaatggtgttgctggtaccgattgtaccaagtcattcaatcgatttattcagttaggttctggaattactcaaactcagcatctatatgtggtgttatacaccagtgttgctctcaaagctgtgctagaacatacagtttatgtcgaagtgtaatctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaactagtggatcctctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaatggcgagtacatggaattggtctggtaagaaaggaagaagaacactacgccgtccttatggtcgaccatacaagtcgtatgttccaacgacgcgagttgttgttcatcaatcagctgttttgaagaaagatgaggttgctggtacagaaatcaagcctgaaggtgatgtagttcgttataaaatgaagaaggtgatgctaacatgtactctgaggatggctcctggagagttagtgaattacgttattgttaaatgtaattcgcctattgttacttggtctgctgcgtttaccagtcctgctctcttggtgaaggagagttgtcaggatatgatcacaattattgcgaaaggcaaggtcgaatcgaatggtgttgctggtaccgattgtaccaagtcattcaatcgatttattcagttaggttctggaattactcaaactcagcatctatatgtggtgttatacaccagtgttgctctcaaagctgtgctagaacatacagtttatgtcgaagtgtaatctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaactagtggatcctctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaatggcgagtacatggaattggtctggtaagaaaggaagaagaacactacgccgtccttatggtcgaccatacaagtcgtatgttccaacgacgcgagttgttgttcatcaatcagctgttttgaagaaagatgaggttgctggtacagaaatcaagcctgaaggtgatgtagttcgttataaaatgaagaaggtgatgctaacatgtactctgaggatggctcctggagagttagtgaattacgttattgttaaatgtaattcgcctattgttacttggtctgctgcgtttaccagtcctgctctcttggtgaaggagagttgtcaggatatgatcacaattattgcgaaaggcaaggtcgaatcgaatggtgttgctggtaccgattgtaccaagtcattcaatcgatttattcagttaggttctggaattactcaaactcagcatctatatgtggtgttatacaccagtgttgctctcaaagctgtgctagaacatacagtttatgtcgaagtgtaatctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaactagtggatcctctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaatggcgagtacatggaattggtctggtaagaaaggaagaagaacactacgccgtccttatggtcgaccatacaagtcgtatgttccaacgacgcgagttgttgttcatcaatcagctgttttgaagaaagatgaggttgctggtacagaaatcaagcctgaaggtgatgtagttcgttataaaatgaagaaggtgatgctaacatgtactctgaggatggctcctggagagttagtgaattacgttattgttaaatgtaattcgcctattgttacttggtctgctgcgtttaccagtcctgctctcttggtgaaggagagttgtcaggatatgatcacaattattgcgaaaggcaaggtcgaatcgaatggtgttgctggtaccgattgtaccaagtcattcaatcgatttattcagttaggttctggaattactcaaactcagcatctatatgtggtgttatacaccagtgttgctctcaaagctgtgctagaacatacagtttatgtcgaagtgtaatctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaactagtggatcctctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaatggcgagtacatggaattggtctggtaagaaaggaagaagaacactacgccgtccttatggtcgaccatacaagtcgtatgttccaacgacgcgagttgttgttcatcaatcagctgttttgaagaaagatgaggttgctggtacagaaatcaagcctgaaggtgatgtagttcgttataaaatgaagaaggtgatgctaacatgtactctgaggatggctcctggagagttagtgaattacgttattgttaaatgtaattcgcctattgttacttggtctgctgcgtttaccagtcctgctctcttggtgaaggagagttgtcaggatatgatcacaattattgcgaaaggcaaggtcgaatcgaatggtgttgctggtaccgattgtaccaagtcattcaatcgatttattcagttaggttctggaattactcaaactcagcatctatatgtggtgttatacaccagtgttgctctcaaagctgtgctagaacatacagtttatgtcgaagtgtaatctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaactagtggatcctctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaatggcgagtacatggaattggtctggtaagaaaggaagaagaacactacgccgtccttatggtcgaccatacaagtcgtatgttccaacgacgcgagttgttgttcatcaatcagctgttttgaagaaagatgaggttgctggtacagaaatcaagcctgaaggtgatgtagttcgttataaaatgaagaaggtgatgctaacatgtactctgaggatggctcctggagagttagtgaattacgttattgttaaatgtaattcgcctattgttacttggtctgctgcgtttaccagtcctgctctcttggtgaaggagagttgtcaggatatgatcacaattattgcgaaaggcaaggtcgaatcgaatggtgttgctggtaccgattgtaccaagtcattcaatcgatttattcagttaggttctggaattactcaaactcagcatctatatgtggtgttatacaccagtgttgctctcaaagctgtgctagaacatacagtttatgtcgaagtgtaatctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaactagtggatcctctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaatggcgagtacatggaattggtctggtaagaaaggaagaagaacactacgccgtccttatggtcgaccatacaagtcgtatgttccaacgacgcgagttgttgttcatcaatcagctgttttgaagaaagatgaggttgctggtacagaaatcaagcctgaaggtgatgtagttcgttataaaatgaagaaggtgatgctaacatgtactctgaggatggctcctggagagttagtgaattacgttattgttaaatgtaattcgcctattgttacttggtctgctgcgtttaccagtcctgctctcttggtgaaggagagttgtcaggatatgatcacaattattgcgaaaggcaaggtcgaatcgaatggtgttgctggtaccgattgtaccaagtcattcaatcgatttattcagttaggttctggaattactcaaactcagcatctatatgtggtgttatacaccagtgttgctctcaaagctgtgctagaacatacagtttatgtcgaagtgtaatctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaactagtggatcctctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaatggcgagtacatggaattggtctggtaagaaaggaagaagaacactacgccgtccttatggtcgaccatacaagtcgtatgttccaacgacgcgagttgttgttcatcaatcagctgttttgaagaaagatgaggttgctggtacagaaatcaagcctgaaggtgatgtagttcgttataaaatgaagaaggtgatgctaacatgtactctgaggatggctcctggagagttagtgaattacgttattgttaaatgtaattcgcctattgttacttggtctgctgcgtttaccagtcctgctctcttggtgaaggagagttgtcaggatatgatcacaattattgcgaaaggcaaggtcgaatcgaatggtgttgctggtaccgattgtaccaagtcattcaatcgatttattcagttaggttctggaattactcaaactcagcatctatatgtggtgttatacaccagtgttgctctcaaagctgtgctagaacatacagtttatgtcgaagtgtaatctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaactagtggatcctctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaatggcgagtacatggaattggtctggtaagaaaggaagaagaacactacgccgtccttatggtcgaccatacaagtcgtatgttccaacgacgcgagttgttgttcatcaatcagctgttttgaagaaagatgaggttgctggtacagaaatcaagcctgaaggtgatgtagttcgttataaaatgaagaaggtgatgctaacatgtactctgaggatggctcctggagagttagtgaattacgttattgttaaatgtaattcgcctattgttacttggtctgctgcgtttaccagtcctgctctcttggtgaaggagagttgtcaggatatgatcacaattattgcgaaaggcaaggtcgaatcgaatggtgttgctggtaccgattgtaccaagtcattcaatcgatttattcagttaggttctggaattactcaaactcagcatctatatgtggtgttatacaccagtgttgctctcaaagctgtgctagaacatacagtttatgtcgaagtgtaatctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaactagtggatcctctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaatggcgagtacatggaattggtctggtaagaaaggaagaagaacactacgccgtccttatggtcgaccatacaagtcgtatgttccaacgacgcgagttgttgttcatcaatcagctgttttgaagaaagatgaggttgctggtacagaaatcaagcctgaaggtgatgtagttcgttataaaatgaagaaggtgatgctaacatgtactctgaggatggctcctggagagttagtgaattacgttattgttaaatgtaattcgcctattgttacttggtctgctgcgtttaccagtcctgctctcttggtgaaggagagttgtcaggatatgatcacaattattgcgaaaggcaaggtcgaatcgaatggtgttgctggtaccgattgtaccaagtcattcaatcgatttattcagttaggttctggaattactcaaactcagcatctatatgtggtgttatacaccagtgttgctctcaaagctgtgctagaacatacagtttatgtcgaagtgtaatctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaactagtggatcctctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaatggcgagtacatggaattggtctggtaagaaaggaagaagaacactacgccgtccttatggtcgaccatacaagtcgtatgttccaacgacgcgagttgttgttcatcaatcagctgttttgaagaaagatgaggttgctggtacagaaatcaagcctgaaggtgatgtagttcgttataaaatgaagaaggtgatgctaacatgtactctgaggatggctcctggagagttagtgaattacgttattgttaaatgtaattcgcctattgttacttggtctgctgcgtttaccagtcctgctctcttggtgaaggagagttgtcaggatatgatcacaattattgcgaaaggcaaggtcgaatcgaatggtgttgctggtaccgattgtaccaagtcattcaatcgatttattcagttaggttctggaattactcaaactcagcatctatatgtggtgttatacaccagtgttgctctcaaagctgtgctagaacatacagtttatgtcgaagtgtaatctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaactagtggatcctctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaatggcgagtacatggaattggtctggtaagaaaggaagaagaacactacgccgtccttatggtcgaccatacaagtcgtatgttccaacgacgcgagttgttgttcatcaatcagctgttttgaagaaagatgaggttgctggtacagaaatcaagcctgaaggtgatgtagttcgttataaaatgaagaaggtgatgctaacatgtactctgaggatggctcctggagagttagtgaattacgttattgttaaatgtaattcgcctattgttacttggtctgctgcgtttaccagtcctgctctcttggtgaaggagagttgtcaggatatgatcacaattattgcgaaaggcaaggtcgaatcgaatggtgttgctggtaccgattgtaccaagtcattcaatcgatttattcagttaggttctggaattactcaaactcagcatctatatgtggtgttatacaccagtgttgctctcaaagctgtgctagaacatacagtttatgtcgaagtgtaatctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaactagtggatcctctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaatggcgagtacatggaattggtctggtaagaaaggaagaagaacactacgccgtccttatggtcgaccatacaagtcgtatgttccaacgacgcgagttgttgttcatcaatcagctgttttgaagaaagatgaggttgctggtacagaaatcaagcctgaaggtgatgtagttcgttataaaatgaagaaggtgatgctaacatgtactctgaggatggctcctggagagttagtgaattacgttattgttaaatgtaattcgcctattgttacttggtctgctgcgtttaccagtcctgctctcttggtgaaggagagttgtcaggatatgatcacaattattgcgaaaggcaaggtcgaatcgaatggtgttgctggtaccgattgtaccaagtcattcaatcgatttattcagttaggttctggaattactcaaactcagcatctatatgtggtgttatacaccagtgttgctctcaaagctgtgctagaacatacagtttatgtcgaagtgtaatctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaactagtggatcctctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaatggcgagtacatggaattggtctggtaagaaaggaagaagaacactacgccgtccttatggtcgaccatacaagtcgtatgttccaacgacgcgagttgttgttcatcaatcagctgttttgaagaaagatgaggttgctggtacagaaatcaagcctgaaggtgatgtagttcgttataaaatgaagaaggtgatgctaacatgtactctgaggatggctcctggagagttagtgaattacgttattgttaaatgtaattcgcctattgttacttggtctgctgcgtttaccagtcctgctctcttggtgaaggagagttgtcaggatatgatcacaattattgcgaaaggcaaggtcgaatcgaatggtgttgctggtaccgattgtaccaagtcattcaatcgatttattcagttaggttctggaattactcaaactcagcatctatatgtggtgttatacaccagtgttgctctcaaagctgtgctagaacatacagtttatgtcgaagtgtaatctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaactagtggatcctctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaatggcgagtacatggaattggtctggtaagaaaggaagaagaacactacgccgtccttatggtcgaccatacaagtcgtatgttccaacgacgcgagttgttgttcatcaatcagctgttttgaagaaagatgaggttgctggtacagaaatcaagcctgaaggtgatgtagttcgttataaaatgaagaaggtgatgctaacatgtactctgaggatggctcctggagagttagtgaattacgttattgttaaatgtaattcgcctattgttacttggtctgctgcgtttaccagtcctgctctcttggtgaaggagagttgtcaggatatgatcacaattattgcgaaaggcaaggtcgaatcgaatggtgttgctggtaccgattgtaccaagtcattcaatcgatttattcagttaggttctggaattactcaaactcagcatctatatgtggtgttatacaccagtgttgctctcaaagctgtgctagaacatacagtttatgtcgaagtgtaatctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaactagtggatcctctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaatggcgagtacatggaattggtctggtaagaaaggaagaagaacactacgccgtccttatggtcgaccatacaagtcgtatgttccaacgacgcgagttgttgttcatcaatcagctgttttgaagaaagatgaggttgctggtacagaaatcaagcctgaaggtgatgtagttcgttataaaatgaagaaggtgatgctaacatgtactctgaggatggctcctggagagttagtgaattacgttattgttaaatgtaattcgcctattgttacttggtctgctgcgtttaccagtcctgctctcttggtgaaggagagttgtcaggatatgatcacaattattgcgaaaggcaaggtcgaatcgaatggtgttgctggtaccgattgtaccaagtcattcaatcgatttattcagttaggttctggaattactcaaactcagcatctatatgtggtgttatacaccagtgttgctctcaaagctgtgctagaacatacagtttatgtcgaagtgtaatctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaactagtggatcctctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaatggcgagtacatggaattggtctggtaagaaaggaagaagaacactacgccgtccttatggtcgaccatacaagtcgtatgttccaacgacgcgagttgttgttcatcaatcagctgttttgaagaaagatgaggttgctggtacagaaatcaagcctgaaggtgatgtagttcgttataaaatgaagaaggtgatgctaacatgtactctgaggatggctcctggagagttagtgaattacgttattgttaaatgtaattcgcctattgttacttggtctgctgcgtttaccagtcctgctctcttggtgaaggagagttgtcaggatatgatcacaattattgcgaaaggcaaggtcgaatcgaatggtgttgctggtaccgattgtaccaagtcattcaatcgatttattcagttaggttctggaattactcaaactcagcatctatatgtggtgttatacaccagtgttgctctcaaagctgtgctagaacatacagtttatgtcgaagtgtaatctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaactagtggatcctctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaatggcgagtacatggaattggtctggtaagaaaggaagaagaacactacgccgtccttatggtcgaccatacaagtcgtatgttccaacgacgcgagttgttgttcatcaatcagctgttttgaagaaagatgaggttgctggtacagaaatcaagcctgaaggtgatgtagttcgttataaaatgaagaaggtgatgctaacatgtactctgaggatggctcctggagagttagtgaattacgttattgttaaatgtaattcgcctattgttacttggtctgctgcgtttaccagtcctgctctcttggtgaaggagagttgtcaggatatgatcacaattattgcgaaaggcaaggtcgaatcgaatggtgttgctggtaccgattgtaccaagtcattcaatcgatttattcagttaggttctggaattactcaaactcagcatctatatgtggtgttatacaccagtgttgctctcaaagctgtgctagaacatacagtttatgtcgaagtgtaatctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaactagtggatcctctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaatggcgagtacatggaattggtctggtaagaaaggaagaagaacactacgccgtccttatggtcgaccatacaagtcgtatgttccaacgacgcgagttgttgttcatcaatcagctgttttgaagaaagatgaggttgctggtacagaaatcaagcctgaaggtgatgtagttcgttataaaatgaagaaggtgatgctaacatgtactctgaggatggctcctggagagttagtgaattacgttattgttaaatgtaattcgcctattgttacttggtctgctgcgtttaccagtcctgctctcttggtgaaggagagttgtcaggatatgatcacaattattgcgaaaggcaaggtcgaatcgaatggtgttgctggtaccgattgtaccaagtcattcaatcgatttattcagttaggttctggaattactcaaactcagcatctatatgtggtgttatacaccagtgttgctctcaaagctgtgctagaacatacagtttatgtcgaagtgtaatctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaactagtggatcctctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaatggcgagtacatggaattggtctggtaagaaaggaagaagaacactacgccgtccttatggtcgaccatacaagtcgtatgttccaacgacgcgagttgttgttcatcaatcagctgttttgaagaaagatgaggttgctggtacagaaatcaagcctgaaggtgatgtagttcgttataaaatgaagaaggtgatgctaacatgtactctgaggatggctcctggagagttagtgaattacgttattgttaaatgtaattcgcctattgttacttggtctgctgcgtttaccagtcctgctctcttggtgaaggagagttgtcaggatatgatcacaattattgcgaaaggcaaggtcgaatcgaatggtgttgctggtaccgattgtaccaagtcattcaatcgatttattcagttaggttctggaattactcaaactcagcatctatatgtggtgttatacaccagtgttgctctcaaagctgtgctagaacatacagtttatgtcgaagtgtaatctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaactagtggatcctctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaatggcgagtacatggaattggtctggtaagaaaggaagaagaacactacgccgtccttatggtcgaccatacaagtcgtatgttccaacgacgcgagttgttgttcatcaatcagctgttttgaagaaagatgaggttgctggtacagaaatcaagcctgaaggtgatgtagttcgttataaaatgaagaaggtgatgctaacatgtactctgaggatggctcctggagagttagtgaattacgttattgttaaatgtaattcgcctattgttacttggtctgctgcgtttaccagtcctgctctcttggtgaaggagagttgtcaggatatgatcacaattattgcgaaaggcaaggtcgaatcgaatggtgttgctggtaccgattgtaccaagtcattcaatcgatttattcagttaggttctggaattactcaaactcagcatctatatgtggtgttatacaccagtgttgctctcaaagctgtgctagaacatacagtttatgtcgaagtgtaatctgttatcaagaacaaaataataataaataaatatgcatttgatcttaatactccgcgtagcgaatatgttatgttgtttatgtttccaagaatacccttcattaatgaaggggttttgtctattatgaccttgtgacgtcacttgatcccttgctgagctggggcggggcttagtattacccccgccccaggttcagcggagtcatacggacgcaaggatagccattggattgtaagacacgtgtacaatcaggatctgtgattcgtgaagcgaatctgacggaagattgtacgcagcttctttgaagcttcgtggtaggccccattatatgctttaagtttactttataaaaagtaaagaaagatgctcttttttgctttattcgttttaagtggagtacagctgtctttgcttcttcccgaagcaaagtatttctctctctataaaagctgcatttagttttcgttgtttcagacaacgaaaactagt

SEQ ID NO: 13 - DNA, 29 bp, synthetic construct
  source 1..29: mol_type = other DNA
ttacaacgaaaatgggtctgaaatatttc

SEQ ID NO: 14 - DNA, 27 bp, synthetic construct
  source 1..27: mol_type = other DNA
gaacaacgaagataaacgcagctttta

SEQ ID NO: 15 - DNA, 21 bp, synthetic construct
  source 1..21: mol_type = other DNA
ctagtcagccatccaatggtg

SEQ ID NO: 16 - DNA, 21 bp, synthetic construct
  source 1..21: mol_type = other DNA
gtgcagggtttgattgtctgc

SEQ ID NO: 17 - DNA, 25 bp, synthetic construct
  source 1..25: mol_type = other DNA
cttcaatggtagtttctatggaggt

SEQ ID NO: 18 - DNA, 24 bp, synthetic construct
  source 1..24: mol_type = other DNA
ctaacactgcaactcacaagcaag

SEQ ID NO: 19 - DNA, 24 bp, synthetic construct
  source 1..24: mol_type = other DNA
gaagaagaggaattgttcatcttc

SEQ ID NO: 20 - DNA, 27 bp, synthetic construct
  source 1..27: mol_type = other DNA
ataaacacagcttttatagacgatgag

SEQ ID NO: 21 - DNA, 23 bp, synthetic construct
  source 1..23: mol_type = other DNA
gattagttccaggagcccatttt

SEQ ID NO: 22 - DNA, 21 bp, synthetic construct
  source 1..21: mol_type = other DNA
gcttcatacctgcaagagacg

SEQ ID NO: 23 - DNA, 24 bp, synthetic construct
  source 1..24: mol_type = other DNA
ggattttaatacgcgtggacgatc

SEQ ID NO: 24 - DNA, 23 bp, synthetic construct
  source 1..23: mol_type = other DNA
aacggctataagtcactccgtac

SEQ ID NO: 25 - DNA, 22 bp, synthetic construct
  source 1..22: mol_type = other DNA
gcgatcatcttcaacaagagcg

SEQ ID NO: 26 - DNA, 25 bp, synthetic construct
  source 1..25: mol_type = other DNA
gaacaatcttctctctcarctcttc

SEQ ID NO: 27 - DNA, 24 bp, synthetic construct
  source 1..24: mol_type = other DNA
gtcctgaaagagaacgtaattggg

SEQ ID NO: 28 - DNA, 24 bp, synthetic construct
  source 1..24: mol_type = other DNA
cagtagcttcaaatggagttctgg

SEQ ID NO: 29 - DNA, 18 bp, synthetic construct
  source 1..18: mol_type = other DNA
cgtattgcttttttaacg

SEQ ID NO: 30 - DNA, 17 bp, synthetic construct
  source 1..17: mol_type = other DNA
cgtgaataaaggtagac

SEQ ID NO: 31 - DNA, 22 bp, synthetic construct
  source 1..22: mol_type = other DNA
gattcgtggcttgttgatgagg

SEQ ID NO: 32 - DNA, 22 bp, synthetic construct
  source 1..22: mol_type = other DNA
gcgaactggtgaaacamccatg

SEQ ID NO: 33 - DNA, 21 bp, synthetic construct
  source 1..21: mol_type = other DNA
acaaggattgaaggtccatgg

SEQ ID NO: 34 - DNA, 23 bp, synthetic construct
  source 1..23: mol_type = other DNA
atcttccttcattgcataagctc

SEQ ID NO: 35 - DNA, 22 bp, synthetic construct
  source 1..22: mol_type = other DNA
ggtaccgattgtaccaagtcat

SEQ ID NO: 36 - DNA, 21 bp, synthetic construct
  source 1..21: mol_type = other DNA
agcaacaccattcgattcgac